SEQUENCE LISTING (63 sequences)

SEQ ID NO: 1 - protein, 14 aa, artificial sequence
  Peptide compound
  MISC_FEATURE (1)..(1): Xaa is absent or 1 to 6 amino acids
  MISC_FEATURE (4)..(4): Xaa is 1 to 5 amino acids
  MISC_FEATURE (7)..(7): Xaa is 1 to 5 amino acids
  MISC_FEATURE (10)..(11): Xaa is 1 to 5 amino acids
  MISC_FEATURE (14)..(14): Xaa is absent or is 1 to 6 amino acids
XWSXWSXCSXXCGX

SEQ ID NO: 2 - protein, 12 aa, Artificial sequence
  Peptide compound
  MISC_FEATURE (3)..(3): Xaa is 1 to 5 amino acids
  MISC_FEATURE (6)..(6): Xaa is 1 to 5 amino acids
  MISC_FEATURE (9)..(10): Xaa is 1 to 5 amino acids
WSXWSXCSXXCG

SEQ ID NO: 3 - protein, 12 aa, Artificial sequence
  Peptide compound
WSGWSSCSRSCG

SEQ ID NO: 4 - protein, 12 aa, Artificial sequence
  Peptide compound
WSSWSGCSRSCG

SEQ ID NO: 5 - protein, 12 aa, artificial sequence
  peptide compound
WSSWGSCSRSCG

SEQ ID NO: 6 - protein, 12 aa, artificial sequence
  peptide compound
WSSWGGCSRSCG

SEQ ID NO: 7 - protein, 12 aa, artificial sequence
  peptide compound
WSSWSSCSRSCG

SEQ ID NO: 8 - protein, 12 aa, artificial sequence
  peptide compound
WSGWSGCSRSCG

SEQ ID NO: 9 - protein, 12 aa, artificial sequence
  peptide compound
WSGWGSCSRSCG

SEQ ID NO: 10 - protein, 12 aa, artificial sequence
  peptide compound
WSGWGGCSRSCG

SEQ ID NO: 11 - protein, 12 aa, artificial sequence
  peptide compound
WSSWSSCSVSCG

SEQ ID NO: 12 - protein, 12 aa, artificial sequence
  peptide compound
WSSWSGCSVSCG

SEQ ID NO: 13 - protein, 12 aa, artificial sequence
  peptide compound
WSSWGSCSVSCG

SEQ ID NO: 14 - protein, 12 aa, artificial sequence
  peptide compound
WSSWGGCSVSCG

SEQ ID NO: 15 - protein, 12 aa, artificial sequence
  peptide compound
WSGWSSCSVSCG

SEQ ID NO: 16 - protein, 12 aa, artificial sequence
  peptide compound
WSGWSGCSVSCG

SEQ ID NO: 17 - protein, 12 aa, artificial sequence
  peptide compound
WSGWGSCSVSCG

SEQ ID NO: 18 - protein, 12 aa, artificial sequence
  peptide compound
WSGWGGCSVSCG

SEQ ID NO: 19 - protein, 12 aa, artificial sequence
  peptide compound
WSSWSSCSVTCG

SEQ ID NO: 20 - protein, 12 aa, artificial sequence
  peptide compound
WSSWSGCSVTCG

SEQ ID NO: 21 - protein, 12 aa, artificial sequence
  peptide compound
WSSWGSCSVTCG

SEQ ID NO: 22 - protein, 12 aa, artificial sequence
  peptide compound
WSSWGGCSVTCG

SEQ ID NO: 23 - protein, 12 aa, artificial sequence
  peptide compound
WSGWSSCSVTCG

SEQ ID NO: 24 - protein, 12 aa, Artificial Sequence
  peptide compound
WSGWSGCSVTCG

SEQ ID NO: 25 - protein, 12 aa, Artificial Sequence
  peptide compound
WSGWGSCSVTCG

SEQ ID NO: 26 - protein, 12 aa, Artificial Sequence
  peptide compound
WSGWGGCSVTCG

SEQ ID NO: 27 - protein, 12 aa, Artificial Sequence
  peptide compound
WSSWSSCSRTCG

SEQ ID NO: 28 - protein, 12 aa, artificial sequence
  peptide compound
WSSWSGCSRTCG

SEQ ID NO: 29 - protein, 12 aa, Artificial Sequence
  peptide compound
WSSWGSCSRTCG

SEQ ID NO: 30 - protein, 12 aa, Artificial Sequence
  peptide compound
WSSWGGCSRTCG

SEQ ID NO: 31 - protein, 12 aa, Artificial Sequence
  peptide compound
WSGWSSCSRTCG

SEQ ID NO: 32 - protein, 12 aa, Artificial Sequence
  peptide compound
WSGWSGCSRTCG

SEQ ID NO: 33 - protein, 12 aa, Artificial Sequence
  peptide compound
WSGWGSCSRTCG

SEQ ID NO: 34 - protein, 12 aa, Artificial Sequence
  peptide compound
WSGWGGCSRTCG

SEQ ID NO: 35 - protein, 13 aa, Artificial Sequence
  peptide compound
PWSGWSSCSRSCG

SEQ ID NO: 36 - protein, 14 aa, Artificial Sequence
  peptide compound
APWSGWSSCSRSCG

SEQ ID NO: 37 - protein, 15 aa, Artificial Sequence
  peptide compound
LAPWSGWSSCSRSCG

SEQ ID NO: 38 - protein, 16 aa, Artificial Sequence
  peptide compound
VLAPWSGWSSCSRSCG

SEQ ID NO: 39 - protein, 13 aa, Artificial Sequence
  peptide compound
WSGWSSCSRSCGL

SEQ ID NO: 40 - protein, 14 aa, Artificial Sequence
  peptide compound
WSGWSSCSRSCGLG

SEQ ID NO: 41 - protein, 15 aa, Artificial Sequence
  peptide compound
WSGWSSCSRSCGLGL

SEQ ID NO: 42 - protein, 16 aa, Artificial Sequence
  peptide compound
WSGWSSCSRSCGLGLI

SEQ ID NO: 43 - protein, 17 aa, Artificial Sequence
  peptide compound
WSGWSSCSRSCGLGLIF

SEQ ID NO: 44 - protein, 14 aa, Artificial Sequence
  peptide compound
PWSGWSSCSRSCGL

SEQ ID NO: 45 - protein, 15 aa, Artificial Sequence
  peptide compound
PWSGWSSCSRSCGLG

SEQ ID NO: 46 - protein, 16 aa, Artificial Sequence
  peptide compound
PWSGWSSCSRSCGLGL

SEQ ID NO: 47 - protein, 17 aa, Artificial Sequence
  peptide compound
PWSGWSSCSRSCGLGLI

SEQ ID NO: 48 - protein, 18 aa, Artificial Sequence
  peptide compound
PWSGWSSCSRSCGLGLIF

SEQ ID NO: 49 - protein, 15 aa, Artificial Sequence
  peptide compound
APWSGWSSCSRSCGL

SEQ ID NO: 50 - protein, 16 aa, Artificial Sequence
  peptide compound
APWSGWSSCSRSCGLG

SEQ ID NO: 51 - protein, 17 aa, Artificial Sequence
  peptide compound
APWSGWSSCSRSCGLGL

SEQ ID NO: 52 - protein, 18 aa, Artificial Sequence
  peptide compound
APWSGWSSCSRSCGLGLI

SEQ ID NO: 53 - protein, 19 aa, Artificial Sequence
  peptide compound
APWSGWSSCSRSCGLGLIF

SEQ ID NO: 54 - protein, 16 aa, Artificial Sequence
  peptide compound
LAPWSGWSSCSRSCGL

SEQ ID NO: 55 - protein, 17 aa, Artificial Sequence
  peptide compound
LAPWSGWSSCSRSCGLG

SEQ ID NO: 56 - protein, 18 aa, Artificial Sequence
  peptide compound
LAPWSGWSSCSRSCGLGL

SEQ ID NO: 57 - protein, 19 aa, Artificial Sequence
  peptide compound
LAPWSGWSSCSRSCGLGLI

SEQ ID NO: 58 - protein, 20 aa, Artificial Sequence
  peptide compound
LAPWSGWSSCSRSCGLGLIF

SEQ ID NO: 59 - protein, 17 aa, Artificial Sequence
  peptide compound
VLAPWSGWSSCSRSCGL

SEQ ID NO: 60 - protein, 18 aa, Artificial Sequence
  peptide compound
VLAPWSGWSSCSRSCGLG

SEQ ID NO: 61 - protein, 19 aa, Artificial Sequence
  peptide compound
VLAPWSGWSSCSRSCGLGL

SEQ ID NO: 62 - protein, 20 aa, Artificial Sequence
  peptide compound
VLAPWSGWSSCSRSCGLGLI

SEQ ID NO: 63 - protein, 21 aa, Artificial Sequence
  peptide compound
VLAPWSGWSSCSRSCGLGLIF

(19) United States
(12) Patent Application Publication
(10) Pub. No.: US 2023/0212225 A1
(43) Pub. Date: Jul. 6, 2023

(54) PEPTIDE COMPOSTIONS AND METHODS FOR TREATING TAUOPATHIES

(71) Applicant: AXOLTIS PHARMA, CLERMONT-FERRAND (FR)

(72) Inventors: Manuel BLANC, Lyon (FR); Yann GODFRIN, CALUIRE (FR); Juliette LE DOUCE, LYON (FR)

(21) Appl. No.: 17/781,214

(22) PCT Filed: Dec. 4, 2020
(86) PCT No.: PCT/EP2020/084746

§ 371 Date: May 31, 2022

(30) Foreign Application Priority Data
Dec. 5, 2019 (EP) 19306582.8
Aug. 12, 2020 (EP) 20305925.8

(51) Int. Cl. C07K 7/08; A61K 9/00; A61P 25/28

(52) U.S. Cl. CPC C07K 7/08; A61K 9/0019; A61P 25/28

(57) ABSTRACT

The invention concerns treating Tauopathies such as Alzheimer’s disease with a SCO- Spondin derived peptide administered through a systemic route to the patient. Said peptide has amino acid sequence X1-W-S-A1-W-S-A2-C-S-A3-A4-C-G-X2, in which A1, A2, A3 and A4 consists of amino acid sequences consisting of 1 to 5 amino acids, X1 and X2 consists of amino acid sequences consisting of 1 to 6 amino acids, or X1 and X2 are absent; it being possible for the N-terminal amino acid to be acetylated, for the C-terminal amino acid to be amidated, or the N-terminal amino acid to be acetylated and the C-terminal amino acid to be amidated.

The present invention relates to peptides, peptides compositions and methods for the treatment of Tauopathies such as Alzheimer’s Disease (AD).

### BACKGROUND OF INVENTION

Tauopathies are a class of neurodegenerative disorders associated with the pathological aggregation of tau protein. Tau is a microtubule associated protein that is abundant in the Central Nervous System and is predominantly expressed in the axons. Tau protein may play a role in stabilizing microtubule networks in neurons. Excessive or abnormal phosphorylation of Tau can result in tau aggregates which are believed to be a major contributor to disease progression.

Tau phosphorylation is a normal metabolic process, critical in controlling tau’s binding to microtubules, and is ongoing within the brain at all times. Tau may be hyperphosphorylated, and may aggregate as detectable fibrillar deposits in tissues, in both aging and neurodegenerative disease.

Tauopathies include for example Alzheimer’s Disease (AD), Progressive Supranuclear Palsy (PSP), Tau positive Fronto-Temporal Dementia such as Pick’s disease, dementia with Lewy bodies, corticobasal degeneration, Niemann-Pick type C disease, chronic traumatic encephalopathy including dementia pugilistica, postencephalitic parkinsonism. In some tauopathies, such as in AD, aggregation of amyloid-β peptides may be observed in addition to tau aggregates.

There are very limited treatment options for tauopathies. Acetylcholinesterase inhibitors (AChEls) (donepezil, galantamine and rivastigmine) are the mainstay of symptomatic treatment for AD, increasing acetylcholine availability by inhibiting its breakdown in the synapse. However, some patients may not respond or lose benefit of treatment over time. In addition, they may experience side-effects. Consequently, there are patients for which Acetylcholinesterase inhibitors are contraindicated. There is thus an important medical need for innovative treatment options for tauopathies.

SCO-Spondin derived peptides have been described for their neuroregenerative properties notably their ability to improve cell survival and neurite outgrowth in vitro. The use of SCO-spondin derived peptides for the treatment of Spinal Cord Injury has been investigated in animal models.

An objective of the invention is to provide for compositions and methods useful for treating a tauopathy and/or a disorder in which tau protein is involved in the pathology.

Another objective of the invention is to provide for compositions and methods useful for treating a tauopathy and/or a disorder in which amyloid-β protein and tau protein are involved in the pathology.

Another objective is to provide for compositions and methods useful for treating Alzheimer’s Disease (AD); Progressive Supranuclear Palsy (PSP); Tau positive Fronto-Temporal Dementia such as Pick’s disease; dementia with Lewy bodies; corticobasal degeneration; Niemann-Pick type C disease; chronic traumatic encephalopathy including dementia pugilistica; postencephalitic parkinsonism.

Another objective of the invention is to provide for compositions and methods for reducing or disrupting tau protein aggregation, reducing tau protein, and/or reducing tau protein hyper- and/or abnormal phosphorylation, in a subject. Still another objective is to provide such compositions and methods that may be beneficial to a subject suffering from a disease or disorder characterized by, or resulting at least in part from, the tau protein, such as an increased or pathological tau protein level, and/or tau protein aggregation, and/or tau protein hyper- and/or abnormal phosphorylation.

Another objective of the invention is to provide for such compositions and methods, which additionally are capable of interfering with amyloid-β plaque formation, of preventing amyloid-β aggregation and/or of de-polymerization of already formed amyloid-β deposits or amyloid-β aggregates.

Another objective of the invention is to provide for compositions and methods compatible with systemic administration of the active principle for the treatment of tauopathies.

Another objective of the invention is to provide for compositions and methods useful in the treatment of a tauopathy in a patient for which Acetylcholinesterase inhibitors are contraindicated.

Another objective of the invention is to provide for compositions and methods useful in the treatment of a tauopathy in a patient in combination with another drug indicated for treating the tauopathy.

### SUMMARY OF THE INVENTION

The inventors made the unexpected finding that SCO-Spondin derived peptides may be useful for the treatment of tauopathies. In particular, the inventors made the unexpected finding that SCO-Spondin derived peptides modulate tau and in particular its phosphorylation, and reduce tau protein accumulation or tau protein aggregates. In addition, the inventors made the demonstration that this beneficial effect could be obtained through systemic administration of the peptides. Further, the inventors made the finding that compositions or methods according to the invention could benefit patients for which treatment with Acetylcholinesterase inhibitors are contraindicated or still in case of resistance to Acetylcholinesterase inhibitors.

Systemic administration is unexpectedly efficient for the peptides of the invention in this particular context. It confers a significant advantage over administration directly at the site of tau deregulation or administration directly into the CSF (intrathecal or intracerebral injection) because systemic administration is safer and more convenient for the patient to be treated. An important or advantageous aspect of this mode of administration is that it renders possible repeated administrations by the health professional over time for a given patient. The peptides may be readily administered through injection or infusion, including via perfusion. The inventors also found that the bioavailability of these peptides or the biological effect at the lesion level after systemic administration does not require administration of unduly high amounts of peptide. Unexpectedly, the peptide (native or unmodified) develops the biological effect after systemic administration. Unexpectedly also, despite a very short half-life of the peptide (about 12 minutes in monkey, about 19 minutes in human), the biological effect in the CNS is observed. Unexpectedly also, despite this very short half-life, the efficiency of the peptides was observed when the peptide was administered every day or every 2 days.

Described herein are compositions comprising SCO-Spondin derived peptide(s) for treating a tauopathy. This use allows reducing the total level of tau protein in a cell, the level of phosphorylated tau protein, tau aggregation, or a combination thereof. The use allows reducing toxic tau oligomers and treat or slow or prevent a tauopathy and the progression thereof.

In this description, unless contraindication, any disclosed feature applies to the different subject matter that are “composition for use”, “method of use”, and “use of said peptide for the manufacture of a medicament”.

In an aspect, the invention relates to a composition comprising at least one SCO-Spondin derived peptide for use, and a method comprising administering at least one SCO-Spondin derived peptide, for treating a tauopathy in a subject. The use and the method may comprise administering to the subject an efficient or sufficient amount of at least one SCO-Spondin derived peptide. In an embodiment, systemic administration of at least one SCO-Spondin derived peptide is made.

In another aspect, the invention relates to a composition comprising at least one SCO-Spondin derived peptide for use, and a method comprising administering at least one SCO-Spondin derived peptide, for reducing or disrupting tau aggregation in a subject, and/or treating a tauopathy in said subject. The use and the method may comprise administering to the subject an efficient or sufficient amount of at least one SCO-Spondin derived peptide. In an embodiment, systemic administration of at least one SCO-Spondin derived peptide is made.

In another aspect, the invention relates to a composition comprising at least one SCO-Spondin derived peptide for use, and a method comprising administering at least one SCO-Spondin derived peptide, for reducing tau protein and/or tau protein hyperphosphorylation in a subject, and/or treating a tauopathy in said subject. The use and the method may comprise administering to the subject an efficient or sufficient amount of at least one SCO-Spondin derived peptide. In an embodiment, systemic administration of at least one SCO-Spondin derived peptide is made.

In another aspect, the invention relates to a composition comprising at least one SCO-Spondin derived peptide for use in treating a tauopathy through systemic administration to a subject. In another aspect, the invention relates to a method for treating a tauopathy comprising administering at least one SCO-Spondin derived peptide through a systemic route to a subject.

In another aspect, the invention relates to a composition comprising at least one SCO-Spondin derived peptide for use, and a method comprising administering at least one SCO-Spondin derived peptide, for treating a subject for which Acetylcholinesterase inhibitors are contraindicated, or not or no more tolerated, or not sufficiently active, or no more active.

In another object, the invention concerns the use of at least one SCO-Spondin derived peptide for the manufacturing of a pharmaceutical composition for treating a tauopathy. In an embodiment, the composition is for a systemic administration. In an embodiment, the composition comprises a pharmaceutical excipient or vehicle for systemic administration.

In some embodiments of these different aspects, the tauopathy is selected from the group consisting of Alzheimer’s Disease (AD); Progressive Supranuclear Palsy (PSP); Tau positive Fronto-Temporal Dementia such as Pick’s disease; dementia with Lewy bodies; corticobasal degeneration; Niemann-Pick type C disease; chronic traumatic encephalopathy including dementia pugilistica; and postencephalitic parkinsonism.

In an embodiment of these different aspects, the tauopathy is AD. The AD may be one for which a treatment which an Acetylcholinesterase inhibitor (e.g. Donepezil) is contraindicated, is not sufficiently active or no more active, the patient developed a resistance thereto, or the patient developed an intolerance thereto.

In an embodiment, the use is additionally capable of interfering with amyloid-β plaque formation, of preventing amyloid-β aggregation and/or of de-polymerization of already formed amyloid-β deposits or amyloid-β aggregates.

In an embodiment, the subject is also treated with a sufficient amount of an acetylcholinesterase inhibitor.

In another object, the invention concerns a combination of at least one SCO-Spondin derived peptide and an acetylcholinesterase inhibitor, preferably DPZ, for use in a method for treating a tauopathy, wherein the peptide is administered through a systemic route to the subject. Administration of both active principles may in particular be separate, simultaneous or sequential.

In another object, the invention concerns a pharmaceutical composition comprising at least one SCO-Spondin derived peptide and an acetylcholinesterase inhibitor, preferably DPZ, and a pharmaceutically acceptable vehicle, carrier or excipient. Preferably, the composition is suitable for administration through a systemic route. In an embodiment, the composition is for use for treating a tauopathy, with said composition being administered through a systemic route to the subject.

The present invention will now be described in more details using non-limiting examples referring to the figures:

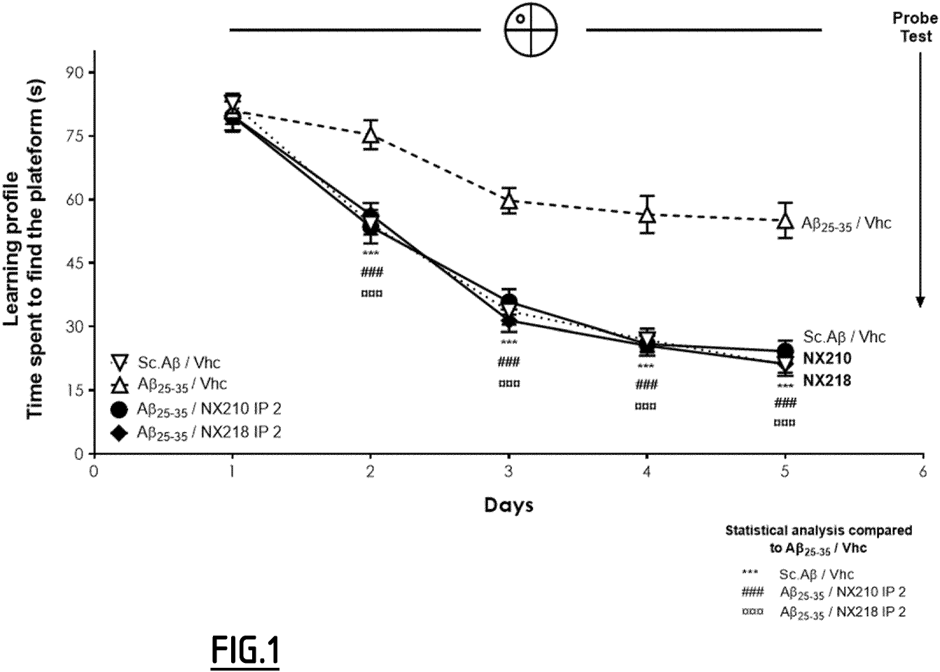

FIG. 1. Effect of IP administrations of NX210 or NX218 (2 mg/kg once a day) on Aβ₂₅₋ ₃₅-induced spatial working memory deficits in mice: time spent to reach the platform was determined during the five training days. Doses are expressed in mg per kg. n is equal to 11-12 per group. Data are expressed as the time spent to find the platform (in sec). ns (non-significant), *** or ### or ¤¤¤ p < 0.001 vs. the Aβ₂₅₋₃₅ / Vhc group, Two-way ANOVA followed by Tukey’s multiple comparison test.

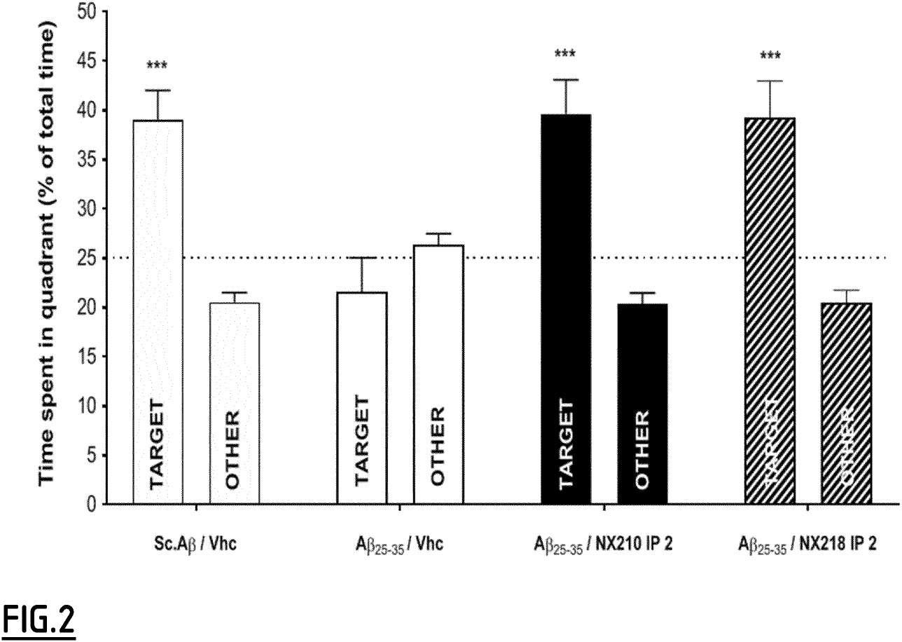

FIG. 2. Effect of IP administrations of NX210 or NX218 (2 mg/kg once a day) on Aβ₂₅₋ ₃₅-induced spatial working memory deficits in mice: time spent in each quadrant was determined during the probe test. Doses are expressed in mg per kg. n is equal to 11-12 per group. Data are expressed as the percentage of time spent in the target quadrant compared to the mean percentage of time spent in the three other quadrants. ns (non-significant), *** p < 0.001 vs. the Aβ₂₅₋₃₅ / Vhc group, Two-way ANOVA followed by Tukey’s multiple comparison test.

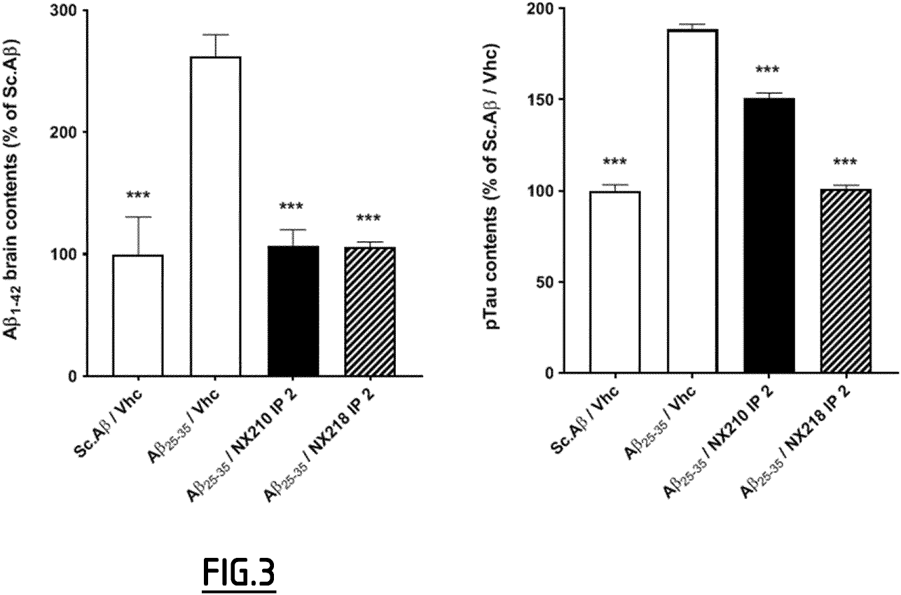

FIG. 3. Effect of IP administrations of NX210 or NX218 (2 mg/kg once a day) on Aβ₂₅₋ ₃₅-induced biochemical changes in brain mice (Aβ₁₋₄₂ and phosphorylated Tau levels). Doses are expressed in mg per kg. n is equal to 5-6 per group. Data are expressed as a percentage of the control group (Sc.Aβ / Vhc group). ns (non-significant), *** p < 0.001 vs. the Aβ₂₅₋₃₅ / Vhc group, One-Way ANOVA followed by Dunnett’s test.

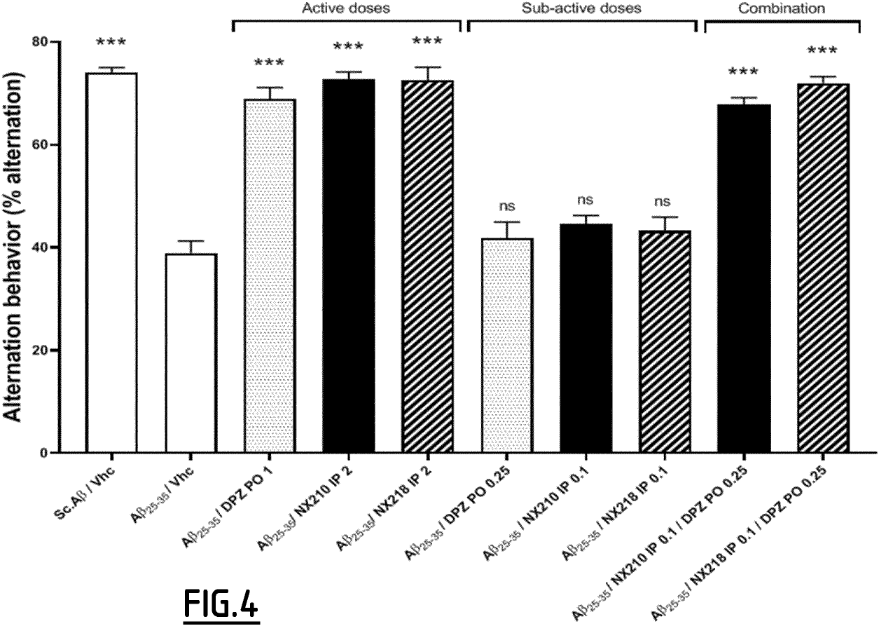

FIG. 4. Effect of administrations of active or subactive doses of NX210 or NX218 (0.1-2 mg/kg once a day, IP), or of DPZ (0.25-1 mg/kg once a day, PO) or combination of subactive doses of NX210 or NX218 with subactive doses of DPZ (0.1 mg/kg for NX210 and NX218, IP, and 0.25 mg/kg for DPZ, PO) on Aβ₂₅₋₃₅-induced short-term memory deficits in mice. Doses are expressed in mg per kg. n is equal to 12 per group. Data are expressed as a percentage of alternation. ns (non-significant), *** p < 0.001 vs. the Aβ₂₅₋₃₅ / Vhc group, One-Way ANOVA followed by Dunnett’s test.

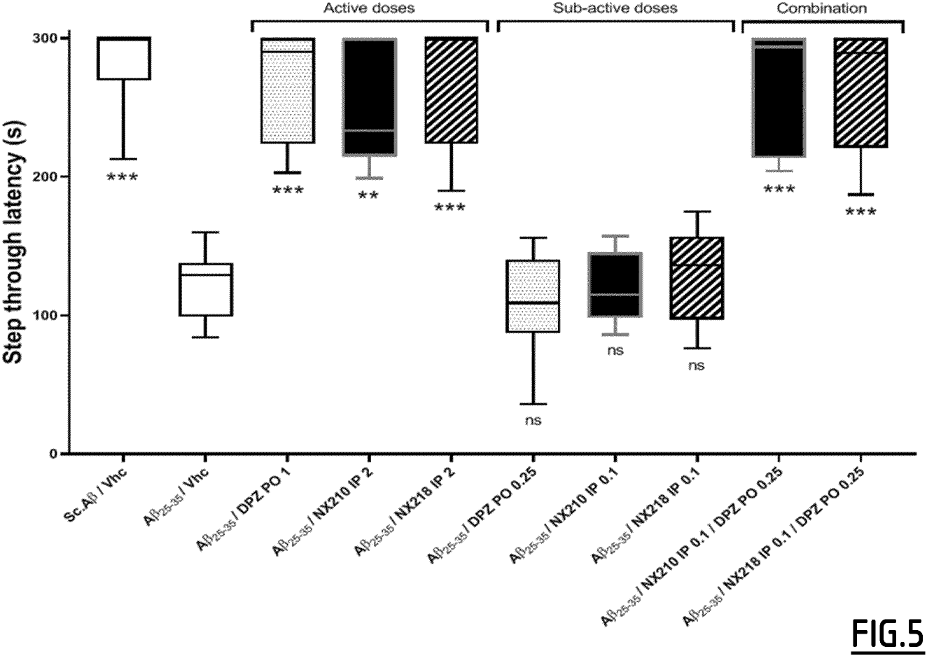

FIG. 5. Effect of administrations of active or subactive doses of NX210 or NX218 (0.1-2 mg/kg once a day, IP), or of DPZ (0.25-1 mg/kg once a day, PO) or combination of subactive doses of NX210 or NX218 with subactive doses of DPZ (0.1 mg/kg for NX210 and NX218, IP, and 0.25 mg/kg for DPZ, PO) on Aβ₂₅₋₃₅-induced long-term memory deficits in mice : step-through latency (STL) measured during the retention session. Doses are expressed in mg per kg. n is equal to 12 per group. Data are expressed in seconds. ns (non-significant), ** p<0.01 and *** p < 0.001 vs. the Aβ₂₅₋₃₅ / Vhc group, Kruskal-Wallis followed by Dunn’s multiple comparison test.

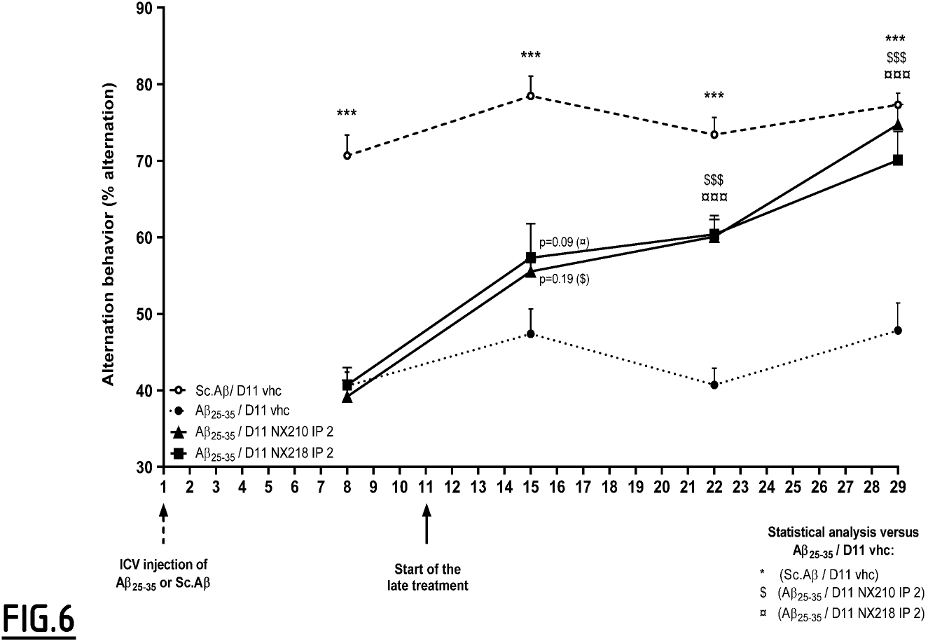

FIG. 6. Effect of administrations of NX210 or NX218 (2 mg/kg once a day started at D11) on Aβ₂₅₋₃₅-induced short-term memory deficits in mice. Doses are expressed in mg per kg. n is equal to 6 per group. Data are expressed as a percentage of alternation over time (D08-15-22-29). ns (non-significant), ***p < 0.001 vs. the Aβ₂₅₋₃₅ / D11 vhc group (* for Sc.Aβ / D11 vhc ; $ for Aβ₂₅₋₃₅ / D11 NX210 IP 2 and ¤ for Aβ₂₅₋₃₅ / D11 NX218 IP 2), One-Way ANOVA followed by Dunnett’s test.

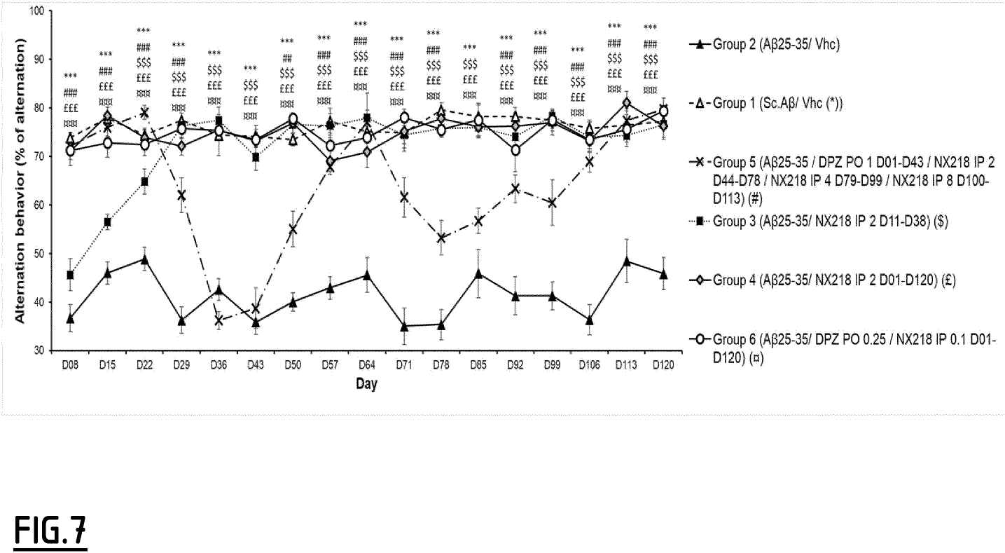

FIG. 7. Long-term effect of administrations of NX218 (2 mg/kg IP once a day for 120 days (group 4)) or DPZ (1 mg/kg per os once a day for 43 days) replaced by increasing doses of NX218 (2 mg/kg IP once a day from day 44 to day 78, then 4 mg/kg once a day from day 79 to day 99 and then 8 mg/kg once a day from day 100 to day 113 (group 5)) or subactive doses of NX218 (0.1 mg/kg IP once a day for 120 days) and DPZ (0.25 mg/kg per os once a day for 120 days) combined (group 6) or transient treatment with NX218 (2 mg/kg IP once a day for 28 days from day 11 to day 38 (group 3)) on Aβ₂₅₋₃₅-induced short-term memory deficits in mice. Data are expressed as a percentage of alternation over time. n is equal to 5-6 per group. ns (non-significant), **p < 0.01, ***p < 0.001 vs. the Aβ₂₅₋₃₅ vhc group (group 2), One-Way ANOVA followed by Dunnett’s test.

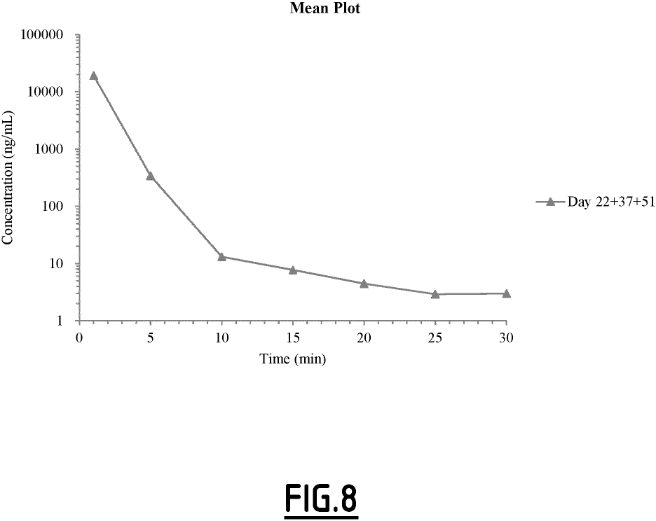

FIG. 8. PK profile after NX210 IV administration in Monkey (bolus IV injection of 10 mg/kg of NX 210). Mean monkey plasma concentration (ng/mL) of NX218 (NX210 cyclic form) is plotted on y-axis with decimal logarithmic scale.

### DETAILED DESCRIPTION

### Tauopathies

The compositions and methods as detailed herein may be used to treat a tauopathy.

In some embodiments, the compositions and methods as detailed herein modulate the tau protein, to treat a tauopathy. Tau is a protein that associates with and stabilizes microtubules. Tau may also be referred to as microtubule associated protein tau (MAPT). Tau proteins may also interact with tubulin to stabilize microtubules and promote tubulin assembly into microtubules. There are six isoforms of tau. Tau proteins are abundant in neurons of the central nervous system and are also expressed at very low levels in central nervous system (CNS) astrocytes and oligodendrocytes. Tau protein may play a role in stabilizing microtubule networks in neurons.

Altered phosphorylation of tau, including excessive phosphorylation (hyperphosphorylation) and abnormal phosphorylation of tau, may result in disruption of microtubule organization, accumulation, and/or aggregation of tau proteins. In some embodiments, tau aggregates do not function properly. For example, tau aggregates may not stabilize microtubules properly. Tau aggregates include, for example, PHF-tau (paired helical filament), NFTs (neurofibrillary tangles), and gliofibrillary tangles. Tau aggregates may also be described as monomeric or high molecular weight multimers and oligomers. Tau aggregates may be insoluble. Tau aggregates may be present in the brain. Tau proteins may be deposited in the form of inclusion bodies within swollen neurons. Aggregation of tau into oligomeric species may lead to various pathologies called tauopathies and may be a major contributor to disease progression.

Tauopathies are a class of neurodegenerative diseases associated with altered phosphorylation of tau and/or pathological aggregation of tau.

The compositions and methods as detailed herein may inhibit or reduce the level of tau protein, inhibit or reduce the level of total tau protein in a cell, inhibit or reduce the level of phosphorylated tau protein, inhibit or reduce or disrupt the aggregation of tau protein, or a combination thereof. The level may be reduced by at least about 10%, at least about 20%, at least about 30%, at least about 40%, at least about 50%, at least about 60%, at least about 70%, at least about 80%, at least about 90%, at least about 95%, or at least about 98%. Tau aggregation may be reduced by at least about 10%, at least about 20%, at least about 30%, at least about 40%, at least about 50%, at least about 60%, at least about 70%, at least about 80%, at least about 90%, at least about 95%, or at least about 98%.

The compositions and methods as detailed herein may inhibit or reduce the level of tau protein, inhibit or reduce the level of total tau protein in a cell, inhibit or reduce the level of phosphorylated tau protein, inhibit or reduce or disrupt the aggregation of tau protein, or a combination thereof, to treat a tauopathy.

“Systemic administration” means any mode of administration or route wherein a substantial part or a sufficient amount of the administered peptide(s) or peptide compound(s) reaches the blood circulation after such administration. Intrathecal administration is excluded as well as any systemic way of administration that does not target the peptide to blood circulation. The systemic administration route of the invention may be qualified of “blood-targeted systemic route of administration”.

Administration or use of “peptide” or “peptides” or “peptide(s), is a generic wording, and the invention encompasses administration or use of one single peptide or more than one single peptide, i.e. the administration or use of at least two peptides according to the present disclosure. Thus, in the present disclosure the singular or the plural is not limited unless indicated to the contrary, and may each time encompass one single peptide, or at least two peptides. The same apply to the equivalent wording “peptide compound” that may be used interchangeably for “peptide”.

“Treating”, “treated”, or “treat”, means delivering an amount of peptide compound according to the invention to a subject. These terms as used herein refers to a therapeutic wherein the object is to slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e. not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment also includes prolonging survival as compared to expected survival if not receiving treatment. The terms “treating”, “treated” or “treat” may include preventing, suppressing, repressing, ameliorating, or completely eliminating the disease. Preventing the disease may involve administering a composition of the present invention to a subject prior to onset of the disease. Suppressing the disease may involve administering a composition of the present invention to a subject after induction of the disease but before its clinical appearance. Repressing or ameliorating the disease may involve administering a composition of the present invention to a subject after clinical appearance of the disease. In an embodiment, beneficial or desired clinical results include a beneficial effect on memory loss and/or cognitive impairments, such as a diminution, a delaying, or a partial or total recovery.

The terms “inhibit” or “inhibiting” mean that an activity is decreased or prevented in the presence of an inhibitor as opposed to in the absence of the inhibitor. The term “inhibition” refers to the reduction or down regulation of a process or the elimination of a stimulus for a process, which results in the absence or minimization of the expression or activity of a biomolecule or polypeptide. Inhibition may be direct or indirect. Inhibition may be specific, that is, the inhibitor inhibits a biomolecule or polypeptide and not others.

“Effective amount”, “sufficient amount”, and similar such as “therapeutically effective amount”, are used interchangeably herein unless otherwise defined, and means a dosage of a peptide or peptides of the invention effective for periods of time necessary to achieve the desired therapeutic result. An effective dosage may be determined by a person skilled in the art and may vary according to factors such as the disease state, age, sex, and weight of the individual, and the ability of the drug to elicit a desired response in the individual. This term as used herein may also refer to an amount effective at bringing about a desired in vivo effect in a subject. A therapeutically effective amount may be administered in one or more administrations (e.g., the composition may be given as a preventative treatment or therapeutically at any stage of disease progression, before or after symptoms, and the like), applications, or dosages, and is not intended to be limited to a particular formulation, combination, or systemic administration route. It is within the scope of the present disclosure that the peptide(s) may be administered at various times during the course of treatment of the subject. The times of administration and dosages used will depend on several factors, such as the goal of treatment (e.g., treating vs. preventing), condition of the subject, etc., and can be readily determined by one skilled in the art. A therapeutically effective amount is also one in which any toxic or detrimental effects of substance are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount. “Effective amount”, “sufficient amount”, may also take into account the combination of different peptides if one considers the amount of the peptides separately, and/or the combination with another active principle, owing to which, for example, the dose of one or the two drugs in the combination may be lowered by result of a combined effect or a synergic effect.

The terms “comprise(s),” “include(s),” “having,” “has,” “can,” “contain(s),” and variants thereof, as used herein, are intended to be open-ended transitional phrases, terms, or words that do not preclude the possibility of additional acts or products, such as peptides, compounds or drugs. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Patient or subject” means an animal, especially a mammal, including a human. In an embodiment, the subject is a human. In other embodiments, the subject is a big or a farm animal, a companion animal (e.g. cat, dog) or a sport animal (e.g. horse).

The “acetylcholinesterase inhibitors” that may be used in combination with the peptides of the invention include donepezil, galantamine and rivastigmine, or any other acetylcholinesterase inhibitor authorized by a Regulatory Authority. In an embodiment such an inhibitor, in particular donepezil, is used in combination with NX210, NX218, or both NX210 and NX218 (SCO-Spondin derived peptides).

As used herein, “simultaneously” or “simultaneous” is used to mean that two agents are administered concurrently, whereas the term “in combination” is used to mean they are administered, if not simultaneously, then “sequentially” within a timeframe that they both are available to act therapeutically within the same time-frame. Thus, administration “sequentially” or “sequential” may permit one agent to be administered within 5 minutes, 10 minutes or a matter of hours after the other provided the circulatory half-life of the first administered agent is such that they are both concurrently present in therapeutically effective amounts. The time delay between administration of the components will vary depending on the exact nature of the components, the interaction therebetween, and their respective half-lives. “Separately” or “separate” is used herein to mean that the gap between administering one agent and the other is significant, i.e. several hours, days, weeks or months, and this may include the case wherein the first administered agent is no longer present in the blood circulation in a therapeutically effective amount when the second agent is administered.

### SCO-Spondin Derived Peptide

“SCO-Spondin” is a glycoprotein specific to the central nervous system and present in all of the vertebrates, from prochordals to humans. It is known as a molecule of extracellular matrices that is secreted by a specific organ located in the roof of the third ventricle, the sub-commissural organ. It is a molecule of large size. It consists of more than 4,500 amino acids and has a multi-modular organization that comprises various preserved protein patterns, including in particular 26 TR or TSR patterns. It is known that certain peptides derived from SCO-Spondin starting from TSR patterns have a biological activity in the nerve or neural cells (in particular described in WO-99/03890).

“TSR or TR patterns” are protein domains of approximately 55-60 residues, based on the alignment of preserved amino acids cysteine, tryptophan and arginine. These patterns were first isolated in TSP-1 (thrombospondin 1) that is a molecule that intervenes in coagulation. They were then described in numerous other molecules such as SCO-Spondin. In fact, this thrombospondin type 1 unit (TSR) comprises, in all the proteins studied so far and previously mentioned, about 55- 60 amino acids (AA) some of which, like cysteine (C), tryptophan (W), serine (S), glycine (G), arginine (R) and proline (P) are highly conserved.

SCO-Spondin peptides or peptide compounds are used in performing the invention (the different objects of the invention, say peptide or composition for use, method of use, method of treatment, use of a peptide for the manufacture of a medicament, etc.).

In particular, the invention uses a peptide of sequence

X1-W-S-A1-W-S-A2-C-S-A3-A4-C-G-X2 (SEQ ID NO: 1)

in which :
A1, A2, A3 and A4 consists of amino acid sequences consisting of 1 to 5 amino acids,
the two cysteines form a disulfide bridge or not,
X1 and X2 consists of amino acid sequences consisting of 1 to 6 amino acids; or X1 and X2 are absent;
it being possible for the N-terminal amino acid to be acetylated (e.g. bears H₃CCOHN-), for the C-terminal amino acid to be amidated (e.g. bears -CONH₂), or both the N-terminal amino acid to be acetylated and the C-terminal amino acid to be amidated.

In an embodiment, in the formula of SEQ ID NO: 1, X1 or X2 or both X1 and X2 are absent. In an embodiment, where X1 and/or X2 is absent, the N-terminal W is acetylated and/or the C-terminal G is amidated. Preferably, both X1 and X2 are absent and the N-terminal is acetylated and the C-terminal G is amidated.

In particular, the invention uses a peptide of sequence

W-S-A1-W-S-A2-C-S-A3-A4-C-G (SEQ ID NO: 2)

in which:
A1, A2, A3 and A4 consists of amino acid sequences consisting of 1 to 5 amino acids,
the two cysteines form a disulfide bridge or not.

In an embodiment of the formulae of SEQ ID NO: 1 and 2, the peptide is a linear peptide, or the cysteines appearing on the peptide formula of SEQ ID NO: 1 and 2 do not form a disulfide bridge (reduced form).

In another embodiment, the two cysteines appearing on the peptide formula of SEQ ID NO: 1 and 2 form a disulfide bridge (oxidized form).

Preferably, in the formulae of SEQ ID NO: 1 and 2, A1, A2, A3 and/or, preferably and A4 consist preferably of 1 or 2 amino acids, more preferably of 1 amino acid.

Preferably, A1 is chosen from G, V, S, P and A, more preferably G, S.

Preferably A2 is chosen from G, V, S, P and A, more preferably G, S.

Preferably, A3 is chosen from R, A and V, more preferably R, V.

Preferably, A4 is chosen from S, T, P and A, more preferably S, T.

Preferably, A1 and A2 are independently chosen from G and S.

Preferably, A3-A4 is chosen from R-S or V-S or V-T or R-T.

Preferably, X1, X2, A1, A2, A3 and A4 do not comprise cysteine.

When X1 is an amino acid sequence of 1 to 6 amino acids, the amino acids are any amino acid, and preferably chosen from V, L, A, P, and any combination thereof.

When X2 is an amino acid sequence of 1 to 6 amino acids, the amino acids are any amino acid, and preferably chosen from L, G, I, F, and any combination thereof.

In an embodiment, the peptide of SEQ ID NO: 1 or 2 is such that A1 and A2 are independently chosen from G and S and A3-A4 is chosen from R-S or V-S or V-T or R-T. In a particular modality, this peptide is further acetylated and/or amidated. In an embodiment, the peptide is a linear peptide, or the cysteines do not form a disulfide bridge. In another embodiment, the peptide has the two cysteines forming a disulfide bridge (C-terminal cyclization). In another embodiment, the peptide as used in the invention or the peptide administered to the patient through a systemic route does comprise both forms, oxidized peptide and linear peptide.

For the purposes of the present invention, the term “amino acids” means both natural amino acids and non-natural amino acids and changes of amino acids, including from natural to non-natural, may be made routinely by the skilled person while keeping the function or efficacy of the original peptide. By “natural amino acids” is meant the amino acids in L form that may be found in natural proteins, i.e. alanine, arginine, asparagine, aspartic acid, cysteine; glutamine, glutamic acid, glycine, histidine, isoleucine, leucine, lysine, methionine, phenylalanine, proline, serine, threonine, tryptophan, tyrosine and valine. By “non-natural amino acid” is meant the preceding amino acids in their D form, as well as the homo forms of certain amino acids such as arginine, lysine, phenylalanine and serine, or the nor forms of leucine or valine. This definition also comprises other amino acids such as alpha-aminobutyric acid, agmatine, alpha-aminoisobutyric acid, sarcosine, statin, ornithine, deaminotyrosine. The nomenclature used to describe the peptide sequences is the international nomenclature using the one-letter code and where the amino-terminal end is shown on the left and the carboxy-terminus is shown on the right. The dashes “-” represent common peptide bonds linking the amino acids of the sequences.

In an embodiment, the peptide according to the invention, for example any one of the peptides of sequence SEQ ID NO: 1-63, comprises an N-terminal acetylation, a C-terminal amidation, or both an N-terminal acetylation and a C-terminal amidation.

In different embodiments, the invention relates to the use of polypeptides consisting essentially of, or consisting of the following amino acid sequences:

TABLE 1

| | |
|---|---|
| W-S-G-W-S-S-C-S-R-S-C-G | SEQ ID NO : 3 |
| W-S-S-W-S-G-C-S-R-S-C-G | SEQ ID NO : 4 |
| W-S-S-W-G-S-C-S-R-S-C-G | SEQ ID NO : 5 |
| W-S-S-W-G-G-C-S-R-S-C-G | SEQ ID NO : 6 |
| W-S-S-W-S-S-C-S-R-S-C-G | SEQ ID NO : 7 |
| W-S-G-W-S-G-C-S-R-S-C-G | SEQ ID NO : 8 |
| W-S-G-W-G-S-C-S-R-S-C-G | SEQ ID NO : 9 |
| W-S-G-W-G-G-C-S-R-S-C-G | SEQ ID NO : 10 |
| W-S-S-W-S-S-C-S-V-S-C-G | SEQ ID NO : 11 |
| W-S-S-W-S-G-C-S-V-S-C-G | SEQ ID NO : 12 |
| W-S-S-W-G-S-C-S-V-S-C-G | SEQ ID NO : 13 |
| W-S-S-W-G-G-C-S-V-S-C-G | SEQ ID NO : 14 |
| W-S-G-W-S-S-C-S-V-S-C-G | SEQ ID NO : 15 |
| W-S-G-W-S-G-C-S-V-S-C-G | SEQ ID NO : 16 |
| W-S-G-W-G-S-C-S-V-S-C-G | SEQ ID NO : 17 |
| W-S-G-W-G-G-C-S-V-S-C-G | SEQ ID NO : 18 |
| W-S-S-W-S-S-C-S-V-T-C-G | SEQ ID NO : 19 |
| W-S-S-W-S-G-C-S-V-T-C-G | SEQ ID NO : 20 |
| W-S-S-W-G-S-C-S-V-T-C-G | SEQ ID NO : 21 |
| W-S-S-W-G-G-C-S-V-T-C-G | SEQ ID NO : 22 |
| W-S-G-W-S-S-C-S-V-T-C-G | SEQ ID NO : 23 |
| W-S-G-W-S-G-C-S-V-T-C-G | SEQ ID NO : 24 |
| W-S-G-W-G-S-C-S-V-T-C-G | SEQ ID NO : 25 |
| W-S-G-W-G-G-C-S-V-T-C-G | SEQ ID NO : 26 |
| W-S-S-W-S-S-C-S-R-T-C-G | SEQ ID NO : 27 |
| W-S-S-W-S-G-C-S-R-T-C-G | SEQ ID NO : 28 |
| W-S-S-W-G-S-C-S-R-T-C-G | SEQ ID NO : 29 |
| W-S-S-W-G-G-C-S-R-T-C-G | SEQ ID NO : 30 |
| W-S-G-W-S-S-C-S-R-T-C-G | SEQ ID NO : 31 |
| W-S-G-W-S-G-C-S-R-T-C-G | SEQ ID NO : 32 |
| W-S-G-W-G-S-C-S-R-T-C-G | SEQ ID NO : 33 |
| W-S-G-W-G-G-C-S-R-T-C-G | SEQ ID NO : 34 |

In an embodiment, the peptides of sequences SEQ ID NO: 3-34 disclosed in Table 1 are linear peptides, or the cysteines do not form a disulfide bridge (reduced peptides). In another embodiment, the peptides of sequences SEQ ID NO: 3-34 disclosed in the preceding table have the two cysteines oxidized to form a disulfide bridge (oxidized peptides). In another embodiment, the peptides as used in the invention or the peptides administered to the patient through a systemic route do comprise both forms, oxidized peptide and linear peptide of the same peptide sequence. In still another embodiment, the peptides as used in the invention or the peptides administered to the patient through a systemic route does comprise a mixture of at least two of these different peptides chosen from sequences SEQ ID NO: 3-34, wherein the mixture may be a mixture of at least two linear peptides or a mixture of at least two oxidized peptides, or a mixture of at least one linear peptide and at least one oxidized peptide, for example having the same amino acid sequence.

In a preferred embodiment, the peptide consists of the amino acid sequence W-S-G-W-S-S-C-S-R-S-C-G (SEQ ID NO: 3). In an embodiment, the peptide is a linear peptide, or the cysteines do not form a disulfide bridge (reduced form called NX210). In another embodiment, the peptides have the two cysteines oxidized to form a disulfide bridge (oxidized form), it is called NX218. In another embodiment, the peptides as used in the invention or the peptides administered to the patient through a systemic route does comprise both forms, oxidized and reduced.

In an embodiment of the peptides of SEQ ID NO: 1,
X1 represents a hydrogen atom or P or A-P or L-A-P or V-L-A-P, and/or
X2 represents a hydrogen atom or L or L-G or L-G-L or L-G-L-I or L-G-L-I-F.

In different embodiments, the invention thus relates to the use of polypeptides consisting or consisting essentially of the following amino acid sequences:

TABLE 2

| | |
|---|---|
| P-W-S-G-W-S-S-C-S-R-S-C-G | SEQ ID NO : 35 |
| A-P-W-S-G-W-S-S-C-S-R-S-C-G | SEQ ID NO : 36 |
| L-A-P-W-S-G-W-S-S-C-S-R-S-C-G | SEQ ID NO : 37 |
| V-L-A-P-W-S-G-W-S-S-C-S-R-S-C-G | SEQ ID NO : 38 |
| W-S-G-W-S-S-C-S-R-S-C-G-L | SEQ ID NO : 39 |
| W-S-G-W-S-S-C-S-R-S-C-G-L-G | SEQ ID NO : 40 |
| W-S-G-W-S-S-C-S-R-S-C-G-L-G-L | SEQ ID NO : 41 |
| W-S-G-W-S-S-C-S-R-S-C-G-L-G-L-I | SEQ ID NO : 42 |
| W-S-G-W-S-S-C-S-R-S-C-G-L-G-L-I-F | SEQ ID NO : 43 |
| P-W-S-G-W-S-S-C-S-R-S-C-G-L | SEQ ID NO : 44 |
| P-W-S-G-W-S-S-C-S-R-S-C-G-L-G | SEQ ID NO : 45 |
| P-W-S-G-W-S-S-C-S-R-S-C-G-L-G-L | SEQ ID NO : 46 |
| P-W-S-G-W-S-S-C-S-R-S-C-G-L-G-L-I | SEQ ID NO : 47 |
| P-W-S-G-W-S-S-C-S-R-S-C-G-L-G-L-I-F | SEQ ID NO : 48 |
| A-P-W-S-G-W-S-S-C-S-R-S-C-G-L | SEQ ID NO : 49 |
| A-P-W-S-G-W-S-S-C-S-R-S-C-G-L-G | SEQ ID NO : 50 |
| A-P-W-S-G-W-S-S-C-S-R-S-C-G-L-G-L | SEQ ID NO : 51 |
| A-P-W-S-G-W-S-S-C-S-R-S-C-G-L-G-L-I | SEQ ID NO : 52 |
| A-P-W-S-G-W-S-S-C-S-R-S-C-G-L-G-L-I-F | SEQ ID NO : 53 |
| L-A-P-W-S-G-W-S-S-C-S-R-S-C-G-L | SEQ ID NO : 54 |
| L-A-P-W-S-G-W-S-S-C-S-R-S-C-G-L-G | SEQ ID NO : 55 |
| L-A-P-W-S-G-W-S-S-C-S-R-S-C-G-L-G-L | SEQ ID NO : 56 |
| L-A-P-W-S-G-W-S-S-C-S-R-S-C-G-L-G-L-I | SEQ ID NO : 57 |
| L-A-P-W-S-G-W-S-S-C-S-R-S-C-G-L-G-L-I-F | SEQ ID NO : 58 |
| V-L-A-P-W-S-G-W-S-S-C-S-R-S-C-G-L | SEQ ID NO : 59 |
| V-L-A-P-W-S-G-W-S-S-C-S-R-S-C-G-L-G | SEQ ID NO : 60 |
| V-L-A-P-W-S-G-W-S-S-C-S-R-S-C-G-L-G-L | SEQ ID NO : 61 |
| V-L-A-P-W-S-G-W-S-S-C-S-R-S-C-G-L-G-L-I | SEQ ID NO : 62 |
| V-L-A-P-W-S-G-W-S-S-C-S-R-S-C-G-L-G-L-I-F | SEQ ID NO : 63 |

In an embodiment, the peptides of sequences SEQ ID NO: 35-63 disclosed in Table 2, or of sequences SEQ ID NO: 3-63 disclosed in Tables 1 + 2, are linear peptides, or the cysteines do not form a disulfide bridge (reduced peptides). In another embodiment, the peptides have the two cysteines oxidized to form a disulfide bridge (oxidized peptides). In another embodiment, the peptides as used in the invention or the peptides administered to the patient through a systemic route does comprise both forms, oxidized peptide and linear peptide of the same peptide sequence. In still another embodiment, the peptides as used in the invention or the peptides administered to the patient through a systemic route does comprise a mixture of at least two of these different peptides chosen from sequences SEQ ID NO: 35-63, or 3-63, wherein the mixture may be a mixture of at least two linear peptides or a mixture of at least two oxidized peptides, or a mixture of at least one linear peptide and at least one oxidized peptide, for example having the same amino acid sequence.

Each one of the peptides of sequences SEQ ID NO: 3-63 may be acetylated, amidated, or acetylated and amidated.

In the present invention, the peptides as used in the invention or the peptides administered to the patient through a systemic route are defined with their amino acid sequences. The peptides as used may be one peptide as disclosed herein, or a mixture of at least two peptides as disclosed herein. The mixtures also encompass the mixture of linear and oxidized peptides, of the same or different amino acid sequences. If a 100% pure peptide may be used, in accordance with the invention, it is possible, and the invention encompasses, that the peptide has a purity greater than 80%, preferably 85%, more preferably 90%, even more preferably equal to or greater than 95, 96, 97, 98, or 99%. Conventional purification methods, for example by chromatography, may be used to purify the desired peptide compound.

In an embodiment, the peptide as used in the invention or the peptide administered to the patient through a systemic route does comprise both forms, oxidized peptide (Op) and linear peptide (Lp), for instance in similar amounts or not, e.g. (% in number) Op: 10, 20, 25, 30, 40, 50, 60, 70, 80, or 90%, the remaining to 100% being the Lp. The oxidized peptide and the linear peptide that are combined may be of the same sequence or of different sequences. For example, the oxidized and linear forms of the peptide of sequence SEQ ID NO: 3 are so combined (NX210 and NX218), for example in the proportions disclosed above. The same apply to any one of the peptides of sequence SEQ ID NO: 4-34 and 35-63.

The pharmaceutical composition as used in the invention comprises as active ingredient a peptide or mixture of peptides as previously described, for example peptides of different amino acid composition or peptides of the same amino acid composition under oxidized and linear forms, and one or more pharmaceutically-acceptable vehicles, carriers or excipients.

The peptide compounds according to the invention may be used in a pharmaceutical composition or in the manufacture of a medicament. In these compositions or medicaments, the active principle may be incorporated into compositions in various forms, i.e. in the form of solutions, generally aqueous solutions, or in freeze-dried form, or in the form of emulsion or any other pharmaceutically and physiologically acceptable form suited to systemic administration route.

In accordance with an important feature of the invention, the peptide compound or the composition containing the same is administered through a systemic route. Mention may be made in particular of the following injection or administration routes: intravenous, intraperitoneal, intranasal, subcutaneous, intramuscular, sublingual, oral, and combinations thereof.

For the intravenous route, one may choose injection, i.e. administration using an injection device (e.g. using syringe, pump) or a perfusion by gravity.

For the subcutaneous route, the composition is administered as a bolus into the subcutis. The sites of injection may be: the outer area of the upper arm; the abdomen, from the rib margin to the iliac crest; the front of the thigh; the back, or the buttock.

For the intranasal or nasal route, one may choose nasal insufflation (the composition is insufflated into the nose, especially using a gas, a powder or vapor), nasal inhalation or nasal instillation.

The compositions containing one or more of the herein-disclosed peptides are sterile. These compositions are suitable for an administration leading to delivering the peptide(s) into the blood circulation. Delivery to the blood circulation is delivery of a sufficient amount of the peptide(s) into the blood circulation, and this sufficient amount is correlated with the beneficial effect in the CNS. In other words, delivery to the blood circulation is delivery of a sufficient amount of the peptide(s) into the blood circulation and of a sufficient “pharmaceutical effect” into the CNS and/or a “cognitive improvement” as defined hereinafter. The “pharmaceutical effect” may comprise inhibiting or reducing the level of tau protein, inhibiting or reducing the level of total tau protein in a cell, inhibiting or reducing the level of phosphorylated tau protein, inhibiting or reducing or disrupting the aggregation of tau protein, or a combination thereof, as disclosed herein. In some embodiments, the active principle in the pharmaceutical composition consists of (1) a linear peptide as disclosed herein, (2) an oxidized peptide as disclosed herein, (3) NX210, (4) NX218 or (5) a mixture of linear and oxidized peptides, such as in particular NX210 and NX218, in similar amounts or not, as disclosed above.

The active principle can be administered in a unit administration form, as a mixture with conventional pharmaceutical supports, carriers, excipients or vehicles, to animals and human beings. Suitable unit administration forms comprise oral-route forms such as tablets, gel capsules, powders, granules and oral suspensions or solutions, sublingual and buccal administration forms, aerosols; implants; subcutaneous, transdermal, topical, intraperitoneal, intramuscular, intravenous, subdermal, transdermal and intranasal administration forms and rectal administration forms.

Preferably, the pharmaceutical compositions contain carriers, excipients or vehicles which are pharmaceutically acceptable for a liquid formulation capable of being injected to deliver the active principle in the blood stream. These may be in particular ready to use solutions, such as isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Sterile injectable solutions are prepared by incorporating the active polypeptides in the required amount in the appropriate solvent followed by filtered sterilization.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For suitable administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. In addition to the compounds of the invention formulated for injection administration, such as intravenous or intramuscular injection, other pharmaceutically acceptable forms include, e.g. tablets or other solids for oral administration; liposomal formulations; time release capsules; and any other form currently used, and delivering the active principle into the blood stream.

One dose expressed in weight of peptide per patient body weight (kg) may range from about 1 µg/kg to about 1 g/kg, in particular from about 10 µg/kg to about 100 mg/kg, e.g. from about 50 µg/kg to about 50 mg/kg.

The dosage regimen may comprise a single administration or repeated administrations. According to an embodiment, repeated administrations may comprise administering one dose per day of treatment, for example one dose every day or every 2 or 3 days over a treatment period. According to another embodiment, repeated administrations may comprise administering at least two doses per day of treatment, for example 2, 3 or more doses per day over a treatment period. In these embodiments, a treatment period may be 1, 2, 3, 4, 5, 6, 7, 8, 9, 10, 15, 20, 25, 30, 35, 40, 45 or more days (e.g. up to 6 months). The treatment is designed so that the patient keeps “benefit” from this treatment over a “period of time”. “Benefit” may comprise the “pharmaceutical effect” mentioned above or a “cognitive improvement”. This “period of time” may depend from the dosage regimen and from the patient itself, e.g. the severity of the illness and the responsiveness of the patient to the regimen dose. “Cognitive improvement” comprises “partial cognitive recovery” and “total cognitive recovery”. The cognitive recovery is said “partial” when in the subject it is observed a partial recovery of the memory alterations; with respect to the initial status of the subject before treatment, there is a significant improvement of the memory, however it remains significantly below with respect to healthy subjects. “Total recovery” means that the subject recovered memory; or that the memory level is not significantly different than healthy subjects.

The cognitive improvement may be measured in a subject, using the MMSE (Mini-Mental State Examination; see Carsten Henneges et al., Journal of Alzheimer’s Disease 52 (2016) 1065-1080) and/or the ADAS-cog (Alzheimer’s Disease Assessment Scale; see Bart Sheehan, Therapeutic Advances in Neurological Disorders 2012, 5(6) 349-358).

MMSE is a brief test consisting of 30 items that has a total score that ranges from normal (30) to severe impairment (0). The questions are grouped into seven categories, with each subscore representing a specific aspect of cognition: orientation in time (score range 0-5); orientation in place (score range 0-5); registration (score range 0-3); attention and concentration (either counting or spelling backwards, score range 0-5); recall (score range 0-3); language (score range 0-8); and drawing (score range 0-1). MMSE criteria (spectrum of AD) that can be used herein: mild AD (MMSE 21-26 points); moderate AD (MMSE 15-20 points); moderately severe/severe (MS/S) AD (MMSE<15 points).

ADAS-cog is widely used to measure cognitive performance in clinical trials and measures multiple cognitive domains, including memory, orientation, visuospatial ability, language, and praxis. Word recall task (score range 0-10); naming objects and fingers (score range 0-5); following commands (score range 0-5); constructional praxis (score range 0-5); ideational praxis (score range 0-5); orientation (score range 0-8); word recognition task (score range 0-12); spoken language ability (score range 0-5); comprehension of spoken language (score range 0-5); word finding difficulty (score range 0-5); and recall of test instructions (score range 0-5).

In an embodiment, the MMSE criteria or the ADAS-cog is measured on a subject before beginning the treatment with a composition as disclosed herein, and then the same subject has this criteria measured once or several (at least two) times after, either during the protocol or after stopping the treatment, or during the protocol, after stopping it and after a lag tie without treatment.

Advantageously, the measurement of the criteria is made in order to decide to keep the treatment ongoing, or to stop the treatment if the criteria shows sufficient cognitive improvement, which may be partial or total improvement, or to renew the treatment if the improvement is lost after a lag period after stopping treatment.

In an embodiment, this “pharmaceutical effect” or “cognitive improvement” lasts a certain period of time, which may be monitored or expectable (for example based on experience or practice). It may be useful in some patients to make sequential such treatments, for example a first treatment, a period without treatment (lag period), and a second or subsequent treatment, and this succession of treatments and lag periods may be multiplied along the life of the patient or as the patient needs it. The dosage regimen may then comprise the initial or first-applied dosage regimen, a period with no treatment (recovery period), a second dosage regimen, a second recovery period; possibly a subsequent third dosage regimen, then a third recovery period, etc. The successive dosage regimens are as disclosed above.

The “period of time” or the lag period (may be called recovery period) during which the patient keeps benefit from a treatment in accordance with a dosage regimen of the invention may be monitored in the patient itself using for example the above described MMSE and ADAS-cog methods, or be based on experience on similar cases. This lag period or recovery period (during which the patient experiences cognitive improvement or another benefit, such as inhibition or reduction of the level of tau protein, the level of total tau protein in a cell, the level of phosphorylated tau protein, or inhibition or reduction or disruption of the aggregation of tau protein, or a combination thereof) may last at least 14 days, or 1, 2, 3, 4, 5, 6 or more months.

In an embodiment a dose is administered by perfusion. Perfusion may last several minutes, tens of minutes, hours, and up to 24 hours a day.

The use according to the invention and the method of treatment of the invention may be characterized as allowing delivery of an amount of peptide compound according to the invention and obtaining a favorable effect on the tauopathy, and/or obtaining one or more of reduction or disruption of tau protein aggregation, reduction of tau protein level, and/or reduction of tau protein hyperphosphorylation, and reduction of abnormal phosphorylation, in a subject.

In an embodiment, the use or the method of treatment has the effect of helping or inducing functional recuperation, meaning that the patient recovers all or part of the functions lost as the result of the tauopathy and/or one or more of tau protein aggregation, tau protein level increase, tau protein hyperphosphorylation, and abnormal phosphorylation.

In an embodiment, the use or the method of treatment has the effect of stopping or inhibiting functional loss due to tauopathy and/or one or more of tau protein aggregation, tau protein level increase, tau protein hyperphosphorylation, and abnormal phosphorylation.

In an embodiment, the invention concerns a combination of at least one SCO-Spondin derived peptide of the invention and an acetylcholinesterase inhibitor, preferably Donepezil (DPZ), for use in a method for treating a tauopathy as disclosed herein, wherein the peptide is administered through a systemic route to the subject. Administration of both active principles may in particular be separate, simultaneous or sequential. The combination of the active principle is capable of having a synergic effect on the treatment efficacy or benefit.

In an embodiment, DPZ is administered to the subject in accordance with Standard treatment, in particular (www.rxlist.com/aricept-druq.htm#indications):
Dosing In Mild To Moderate Alzheimer’s Disease: The recommended starting dosage of ARICEPT® is 5 mg administered once per day in the evening, just prior to retiring. The maximum recommended dosage of ARICEPT® in patients with mild to moderate Alzheimer’s disease is 10 mg per day. A dose of 10 mg should not be administered until patients have been on a daily dose of 5 mg for 4 to 6 weeks.
Dosing In Moderate To Severe Alzheimer’s Disease: The recommended starting dosage of ARICEPT® is 5 mg administered once per day in the evening, just prior to retiring. The maximum recommended dosage of ARICEPT® in patients with moderate to severe Alzheimer’s disease is 23 mg per day. A dose of 10 mg should not be administered until patients have been on a daily dose of 5 mg for 4 to 6 weeks. A dose of 23 mg per day should not be administered until patients have been on a daily dose of 10 mg for at least 3 months.

In case of synergy, for example with DPZ and a peptide of the invention, e.g. NX210, NX218 or NX210 + NX218, the doses of acetylcholinesterase inhibitor, such as DPZ, and/or the dose of peptide may be reduced with respect to the dose for a single molecule. Doses of DPZ may thus be lowered with respect to standard treatment when DPZ and a peptide of the invention are administered to the same subject (simultaneously or sequentially). Doses (subdoses) of DPZ may for example be equal or lower to 3 mg/day, in particular equal or lower to 2.5, 2, 1.5 or 1 mg/day. The dosage or dose regimen may be 0.5 to 3, 0.5 to 2.5, 0.5 to 2, 0.5 to 1.5, or 0.5 to 1 mg/day. In these intervals, the lowest value 0.5 may be replaced by 0.6, 0.7, 0.8, 0.9 or 1).

In an embodiment, the SCO-Spondin derived peptide of the invention and an acetylcholinesterase inhibitor, preferably Donepezil (DPZ), are used separately in the same patient. In particular, the SCO-Spondin derived peptide of the invention is used as a second line treatment of a tauopathy in a patient, wherein the patient has been first treated with an acetylcholinesterase inhibitor, preferably DPZ, as a first line treatment. This first line treatment may be standard, see above. The invention thus concerns a combination of at least one SCO-Spondin derived peptide of the invention and an acetylcholinesterase inhibitor, preferably DPZ, for use in a method for treating a tauopathy as disclosed herein, wherein the peptide is administered through a systemic route to a subject or patient previously treated with said inhibitor.

As an alternative, the second line treatment is made with a combination of the SCO-Spondin derived peptide of the invention and an acetylcholinesterase inhibitor, preferably Donepezil (DPZ). The “combination” may include simultaneous or sequential administration of the agents. In an embodiment, subdoses of the SCO-Spondin derived peptide of the invention and/or of the acetylcholinesterase inhibitor, preferably Donepezil (DPZ), are used.

The method may be further described as follows:
Selecting a patient that has received a therapeutic treatment with said acetylcholinesterase inhibitor; preferably, the patient was treated in accordance with a therapeutic protocol using the inhibitor (preferably a standard or authorized therapeutic protocol);
Optionally determining that the patient experiences a phase wherein he/she does no more respond to the treatment and/or develop an intolerance to said treatment;
Submitting said patient to a therapeutic treatment or protocol of the invention, using a composition according to the invention, or a combination as defined just above;
this treatment may comprise at least one dosage regimen as disclosed above; as disclosed above, the dosage regimen may be followed by a recovery period; and possibly a second dosage regimen, a second recovery period, etc.

In an embodiment, the method comprises:
Submitting a patient in need thereof to a therapeutic treatment with said acetylcholinesterase inhibitor; preferably, the patient is treated in accordance with a therapeutic protocol using the inhibitor;
Optionally determining that the patient experiences a phase wherein he/she does no more respond to the treatment and/or develop an intolerance to said treatment;
Submitting said patient to a therapeutic treatment or protocol of the invention, using a composition according to the invention, or a combination as defined just above; this treatment may comprise at least one dosage regimen as disclosed above; as disclosed above, the dosage regimen may be followed by a recovery period; and possibly a second dosage regimen, a second recovery period, etc.

For reasons of completeness, various aspects of the invention are set out in the following numbered clauses, and the recited features also apply to the other aspects of the invention, say the “for use” and the “use for the facture of a medicament”:

Clause 1. A method of treating a tauopathy in a subject, the method comprising administering to the subject through a systemic route a therapeutic amount of a SCO-Spondin derived peptide and a pharmaceutically acceptable vehicle or excipient.

Clause 2. A method of reducing or disrupting tau aggregation in a subject, the method comprising administering to the subject through a systemic route a therapeutic amount of a SCO-Spondin derived peptide and a pharmaceutically acceptable vehicle or excipient.

Clause 3. A method of reducing tau protein in a subject, the method comprising administering to the subject through a systemic route a therapeutic amount of a SCO-Spondin derived peptide and a pharmaceutically acceptable vehicle or excipient.

Clause 4. The method of any one of the preceding clauses, the method comprising at least one treatment period and at least one recovery period, wherein a treatment period comprises administering to the subject at least one dosage regimen, and a recovery period is a period with no treatment.

Clause 5. The method of any one of the preceding clauses, wherein the level of phosphorylated tau protein is reduced.

Clause 6. The method of any one of the preceding clauses, wherein the level of total tau protein is reduced.

Clause 7. The method of clause 5 or 6, wherein the level is reduced a least 10%.

Clause 8. The method of clause 7, wherein the level is reduced at least 50%.

Clause 9. The method of clause 8, wherein the level is reduced at least 80%.

Clause 10. The method of any one of the preceding clauses, wherein tau aggregation is reduced.

Clause 11. The method of clause 10, wherein tau aggregation is reduced a least 10%.

Clause 12. The method of clause 11, wherein tau aggregation is reduced at least 50%.

Clause 13. The method of clause 12, wherein tau aggregation is reduced at least 80%.

Clause 14. The method of any one of the preceding clauses, wherein cognitive improvement is obtained.

Clause 15. The method of any one of clauses 1 to 14, wherein the SCO-spondin derived peptide is selected from the group consisting of the peptides of sequence SEQ ID NO: 1 or 2.

Clause 16. The method of any one of clauses 1 to 14, wherein the SCO-spondin derived peptide is selected from the group consisting of the peptides of sequence SEQ ID NO: 3-63.

Clause 17. The method of any one of the preceding clauses, further comprising administering to the subject a sufficient amount of an acetylcholinesterase inhibitor.

Clause 18. The method of clause 17, wherein the acetylcholinesterase inhibitor is DPZ.

Clause 19. A method of (1) treating a tauopathy in a subject, (2) of reducing or disrupting tau aggregation in a subject, or (3) of reducing tau protein in a subject, the method comprising
Selecting a patient that has received a therapeutic treatment with an acetylcholinesterase inhibitor, preferably DPZ; then
Optionally determining that the patient experiences a phase wherein he/she does no more respond to the treatment and/or develop an intolerance to said treatment;
Submitting said patient to a therapeutic treatment comprising administering to the subject through a systemic route a therapeutic amount of a SCO-Spondin derived peptide and a pharmaceutically acceptable vehicle or excipient, or a combination as defined above.

Clause 20. A method of (1) treating a tauopathy in a subject, (2) of reducing or disrupting tau aggregation in a subject, or (3) of reducing tau protein in a subject, the method comprising
Submitting a patient in need thereof to a therapeutic treatment with an acetylcholinesterase inhibitor, preferably DPZ; then
Optionally determining that the patient experiences a phase wherein he/she does no more respond to the treatment and/or develop an intolerance to said treatment;
Submitting said patient to a therapeutic treatment comprising administering to the subject through a systemic route a therapeutic amount of a SCO-Spondin derived peptide and a pharmaceutically acceptable vehicle or excipient, or a combination as defined above.

Clause 21. The method of any one of clauses 1 to 18, comprising:
determining an initial cognitive status of a patient, e.g. using the MMSE and/or the ADAS-cog method,
submitting said patient to a therapeutic treatment comprising administering to the subject through a systemic route a therapeutic amount of a SCO-Spondin derived peptide and a pharmaceutically acceptable vehicle or excipient, or a combination as defined above,
during the therapeutic treatment, determining a treatment course cognitive status of a patient, e.g. using the same method, MMSE and/or the ADAS-cog method,
comparing the treatment course cognitive treatment status to the initial cognitive status of said patient
optionally, deciding stopping the treatment in case of cognitive partial or total improvement, or pursuing the treatment in case of no or insufficient (partial or total improvement not attained) cognitive improvement.

Clause 22. The method of clause 21, wherein decision was made pursuing treatment, further comprising
during the therapeutic treatment, determining a treatment course cognitive status of a patient, e.g. using the same method, MMSE and/or the ADAS-cog method,
comparing the treatment course cognitive treatment status to the initial cognitive status of said patient
optionally deciding stopping the treatment in case of cognitive partial or total improvement, or pursuing the treatment in case of no or insufficient (partial or total improvement not attained) cognitive improvement.

Clause 23. The method of clause 21 or 22, wherein decision was made stopping the treatment, further comprising
after a period of no treatment, determining an additional cognitive status of said patient, e.g. using the MMSE and/or the ADAS-cog method,
comparing the additional cognitive treatment status to the initial cognitive status of said patient and/or to the treatment course cognitive status
optionally deciding doing nothing in case the status is still better than the initial one or not significantly different than the status at the time of treatment stop; or deciding an additional treatment period according to the invention in the contrary case.

Clause 24. The method of any one of the preceding clauses, wherein the tauopathy is selected from the group consisting of Alzheimer’s Disease (AD); Progressive Supranuclear Palsy (PSP); Tau positive Fronto-Temporal Dementia such as Pick’s disease; dementia with Lewy bodies; corticobasal degeneration; Niemann-Pick type C disease; chronic traumatic encephalopathy including dementia pugilistica; and postencephalitic parkinsonism.

Clause 25. The method of any one of the preceding clauses, wherein the tauopathy is Alzheimer’s disease (AD).

Clause 26. The method of any one of the preceding clauses, wherein the SCO-spondin derived peptide is administered to the subject intravenously, intraarterially, or intraperitoneally.

Clause 27. The method of any one of the preceding clauses, wherein amyloid-β plaque formation is inhibited or reduced, amyloid-β aggregation is prevented or reduced and/or already formed amyloid-β deposits or amyloid-β aggregates are de-polymerized.

Clause 28. The method of any one of the preceding clauses, wherein inhibiting or reducing Amyloid βeta₁₋₄₂, hyperphosphorylation of Tau protein is inhibited or reduced.

Clause 29. The method of any one of the preceding clauses, wherein the peptide is NX210, NX218 or a mixture of NX210 and NX218.

The present invention will now be described in more details using non-limiting examples reffering to the figures:

FIG. 1. Effect of IP administrations of NX210 or NX218 (2 mg/kg once a day) on Aβ_{25- 35}-induced spatial working memory deficits in mice: time spent to reach the platform was determined during the five training days. Doses are expressed in mg per kg. n is equal to 11- 12 per group. Data are expressed as the time spent to find the platform (in sec). ns (non- significant), *** or ### or ¤¤¤ p <0.0001 vs. the Aβ₂₅₋₃₅ /Vhc group, Two-way ANOVA followed by Turkey’s multiple comparison test.

FIG. 2. Effect of IP administrations of NX210 or NX218 (2 mg/kg once a day) on Aβ_{25- 35}-induced spatial working memory deficits in mice: time spent in each quadrant was determinded during the probe test. Doses are expressed in mg per kg. n is equal to 11-12 per group. Data are expressed as the percentage of time spent in the target quadrant compared to the mean percentage of time spent in the three other quadrants. ns (non-significant), *** p < 0.001 vs. the Aβ₂₅₋₃₅ / Vhc group, Two-way ANOVA followed by Turkey’s multiple comparison test.

FIG. 3. Effect of IP administrations of NX210 or NX218 (2 mg/kg once a day) on Aβ_{25- 35}-induced biochemical changes in brain mice (Aβ₁₋₄₂ and phosphorylated Tau levels). Doses are expressed in mg per kg. n is equal to 5-6 per group. Data are expressed as a percentage of the control group (Sc.Aβ / Vhc group). ns (non-significant), *** p < 0.001 vs. the Aβ₂₅₋₃₅ / Vhc group, One-Way ANOVA followed by Dunnett’s test.

FIG. 4. Effect of administrations of active or subactive doses of NX210 or NX218 (0.1-2 mg/kg once a day, IP), or of DPZ (0.25-1 mg/kg once a day, PO) or combination of subactive doses of NX210 or NX218 with subactive doses of DPZ (0.1 mg/kg for NX210 and NX218, IP, and 0.25 mg/kg for DPZ, PO) on Aβ25-35-induced short-term memory deficits in mice. Doses are expressed in mg per kg. n is equal to 12 per group. Data are expressed as a percentage of alternation. ns (non-significant), *** p < 0.001 vs. the Aβ₂₅₋₃₅ / Vhc group, One- Way ANOVA followed by Dunnett’s test.

FIG. 5. Effect of administrations of active subactive doses of NX210 or NX218 (0.1-2 mg/kg once per day, IP), or of DPZ (0.25-1 mg/kg once a day, PO) or combination of subactive doses of NX210 or NX218 with subactive doses of DPZ (0.1 mg/kg for NX210 and NX218, IP, and 0.25 mg/kg for DPZ, PO) on Aβ₂₅₋₃₅-induced long-term memory deficits in mice : step through latency (STL) measured during the retention session. Doses are expressed in mg per kg. n is equal to 12 per group. Data are expressed in seconds. ns (non-significant), ** p<0.01 and ***p < 0.001 vs. the Aβ25-35 / Vhc group, Kruskal-Wallis followed by Dunn’s multiple comparison test.

FIG. 6. Effect of administrations of NX210 or NX218 (2 mg/kg once a day started at D11) on Aβ25-35-induced short-term memory deficits in mice. Doses are expressed in mg per kg. n is equal to 6 per group. Data are expressed as a percentage of alternation over time (DOB-15-22-29). ns (non-significant), ***p < 0.001 vs. the Aβ₂₅₋₃₅/ D11 vhc group (*for Sc.Aβ / D11 vhc ; $ for Aβ₂₅₋₃₅ / D11 NX210 IP 2 and ¤ for Aβ₂₅₋₃₅ / D11 NX218 IP 2), One-Way ANOVA followed by Dunnett’s test.

FIG. 7. Long-term effect of administrations of NX218 (2 mg/kg IP once a day for 120 days (group 4)) or DPZ (1 mg/kg per os once a day for 43 days) replaced by increasing doses of NX218 (2 mg/kg IP once a day from day 44 to day 78, then 4 mg/kg once a day from day 79 to 99 amd tjem 8mg/kg once a day from day 100 to day 113 (group 5)) or subactive doses of NX218 (0.1 mg/kg IP once a day for 120 days) and DPZ (0.25 mg/kg per os once a day for 28 days from day 11 to day 38 (group 3)) on Aβ₂₅₋₃₅-induced short-term memory deficits in mice. Data are expressed as a percentage of alternation over time. n is equal to 5-6 per group. ns (non-significant), **p < 0.01, ***p < 0.001 vs. the Aβ₂₅₋₃₅ vhc group (group 2), One- Way ANOVA followed by Dunnett’s test.

FIG. 8. PK profile after NX210 IV administration in Monkey (bolus IV injection of 10 mg/kg of NX210). Mean monkey plasma concentration (ng/mL) of NX218 (NX210 cyclic form) is plotted on y-axis with decimal logarithmic scale.

### PART EXAMPLES

### Introduction

Alzheimer’s disease (AD) and related Tauopathies are the most prevalent age-related neurodegenerative disorders, yet with no curative treatments. Prescribed drugs only temporarily slow down dementia symptoms but fail to alter the disease course. Consequently, there is an urgent need to identify new treatments.

The examples are using a model of Tauopathies. In this model, mice were injected intracerebroventricularly (ICV) at Day 01 (D01) with Aβ₂₅₋₃₅ peptide (or with scrambled-Aβ peptide (Sc.Aβ) for Sham animals). This severe animal model recapitulates modulations of specific biomarkers (including deposits of brain Amyloid Beta₁₋₄₂ and hyperphosphorylated Tau protein) involved in the physiopathology and the progression of human Tauopathies, as well as strong cognitive deficits (memory loss) as encountered by the patients (Maurice et al., 1996, 1998, 2013 ; Meunier et al., 2006).

In this dose-response study involving SCO-spondin derived peptides, treatment was started either one (1) hour (as a model of early-stage of the disease) or ten (10) days (as a model of late-stage of the disease) after Aβ₂₅₋₃₅ peptide injection with NX210, NX218 or vehicle (Vhc, H₂O). NX210, NX218 and vehicle were administrated intraperitoneally (IP) either once a day or every other day (once every two days). Synthesis of NX210 and NX218 are described in Examples 1 and 2.

In addition, a combination of NX210 or NX218 with the standard of care was performed (an inhibitor of acetylcholine esterase, here Donepezil, DPZ). To do so, subactive doses of NX210 or NX218 peptides were combined to subactive doses of DPZ in order to study potential synergistic therapeutic effects.

Finally, a study evaluating the long-term effects of treatments based on NX218 and/or DPZ was conducted.

### Example 1: Synthesis of NX Peptides

The manufacturing process of the peptides of sequence SEQ ID NO: 1, 2, or of any of the sequences 3-63, and especially those used in the Part Example, such as NX210 (SEQ ID NO: 3), is based on Solid-Phase Peptide Synthesis applying N-α-Fmoc (side chain) protected amino acids as building blocks in the assembly of the peptide. The protocol employed consists of a coupling of the C-terminal Glycine N-α-Fmoc-protected amino acid bound to an MPPA linker on the MBHA resin, followed by Fmoc coupling / deprotection sequences. After assembly of the peptide on the resin, a step of simultaneous cleavage of the peptide from the resin and deprotection of the side chains of amino acid is carried-out.

The crude peptide is precipitated, filtered and dried. Prior to purification by preparative reverse phase chromatography, the peptide is dissolved in an aqueous solution containing acetonitrile. The purified peptide in solution is the concentrated before undergoing an ion exchange step to obtain the peptide in the form of its acetate salt.

The skilled person may refer for further detail of synthesis to US 6,995,140 and WO2018146283, and for the oxidized forms of the peptides disclosed herein, to WO 2017/051135, all incorporated herein by reference.

The skilled person further has access to the standard methods to produce any of the disclosed peptides of the invention including the N-ter and C-ter modified or protected peptides. Concerning the acetylation and/or the amidation of the peptides at the N-terminal and C-terminal respectively, the skilled person may refer to standard techniques, e.g. those described in Biophysical Journal Volume 95 Nov. 2008 4879-4889, also incorporated by reference.

### Example 2: Synthesis of Cyclic NX Peptides

The polypeptide of sequence W-S-G-W-S-S-C-S-R-S-C-G was added to Human Serum Albumin (HSA) in a 1:1 ratio and incubated for 1 to 3 hours with stirring in air at room temperature. By using HPLC, we observed the formation of a peak corresponding to the polypeptide sequence W-S-G-W-S-S-C-S-R-S-C-G in which the 2 cysteines are linked by a disulfide bridge. After removal of the albumin by precipitation, the product was then purified and analyzed by HPLC. The use of a different ratio of albumin and of polypeptide corresponding to the sequence W-S-G-W-S-S-C-S-R-S-C-G makes it possible to influence the cyclization rate and the final yield of cyclisation, while knowing that a smaller amount of albumin is easier to eliminate. Cyclized compound is NX218.

The skilled person may refer for further detail of synthesis to WO2017051135. This document is incorporated herein by reference.

### Example 3: Dose-Response Efficacy of NX210 and NX218

### Cognitive Assessments

### a. Spontaneous Alternation Performance (Y-Maze)

Seven days after ICV injection of Aβ₂₅₋₃₅ peptide (D08), all animals were tested for spontaneous alternation performance in the Y-maze, an index of spatial working memory (short-term memory test). Each mouse was placed at the end of one arm and allowed to move freely through the maze during an 8 min session. An alternation is defined as entries into all three arms on consecutive occasions. The number of maximum alternations is therefore the total number of arm entries minus two and the percentage of alternation is calculated as (actual alternations / maximum alternations) x 100. This parameter includes the percentage of alternation (memory index) (Maurice et al., 1996, 1998; Meunier et al., 2006).

TABLE 1

| Groups | Y-maze | | | | | |
|---|---|---|---|---|---|---|
| | Mean | SEM | % | n | Significance vs Aβ₂₅₋₃₅ / Vhc | p-value |
| Sc.Aβ / Vhc | 74.04 | 1.00 | 100 | 12 | *** | <0.001 |
| Aβ₂₅₋₃₅ / VhC | 38.95 | 2.34 | 53 | 12 | | |
| Aβ₂₅₋₃₅ / NX210 IP 0.1 | 44.52 | 1.70 | 60 | 12 | ns | 0.3059 |
| Aβ₂₅₋₃₅ / NX210 IP 1 | 58.16 | 2.00 | 79 | 12 | *** | <0.001 |
| Aβ₂₅₋₃₅ / NX210 IP ⅟2d 2 | 74.35 | 1.82 | 100 | 12 | *** | <0.001 |
| Aβ₂₅₋₃₅ / NX210 IP 2 | 72.67 | 1.48 | 98 | 12 | *** | <0.001 |
| Aβ₂₅₋₃₅ / NX210 IP ⅟2d 3.75 | 72.62 | 2.44 | 98 | 12 | *** | <0.001 |
| Aβ₂₅₋₃₅ / NX210 IP 3.75 | 70.90 | 1.84 | 96 | 12 | *** | <0.001 |
| Aβ₂₅₋₃₅ / NX218 IP 0.1 | 43.27 | 2.66 | 58 | 12 | ns | 0.6135 |
| Aβ₂₅₋₃₅ / NX218 IP 1 | 70.12 | 1.58 | 95 | 12 | *** | <0.001 |
| Aβ₂₅₋₃₅ / NX218 IP ⅟2d 2 | 72.03 | 1.92 | 97 | 12 | *** | <0.001 |
| Aβ₂₅₋₃₅ / NX218 IP 2 | 72.53 | 2.51 | 98 | 12 | *** | <0.001 |
| Aβ₂₅₋₃₅ / NX218 IP 3.75 | 71.56 | 1.73 | 97 | 12 | *** | <0.001 |

Effect of IP administrations of NX210 or NX218 peptides (0.1-1-2-3.75 mg/kg either once a day or every other day (1/2d)) on Aβ₂₅₋₃₅-induced short-term memory deficits in mice. Doses are expressed in mg per kg. n is equal to 12 per group. Data are expressed as a percentage of alternation. ns (non-significant), *** p < 0.001 vs. the Aβ₂₅₋₃₅ / Vhc group, One-Way ANOVA followed by Dunnett’s test.

The administrations once a day or every other day of NX210 (1, 2 and 3.75 mg/kg) or NX218 (1, 2 and 3.75 mg/kg) significantly rescued spatial short-term working memory deficits. A complete restoration was achieved for NX210 at 2 and 3.75 mg/kg and for NX218 at 1, 2 and 3.75 mg/kg, at levels similar to the non-injured Sham animals (Sc.Aβ / Vhc). This demonstrates the strong efficacy of both NX210 and NX218 peptides in counteracting cognitive deficits (short-term memory loss) observed in this animal model of AD/Tauopathies (Table 1).

The administrations once a day of NX210 (0.1 mg/kg) or NX218 (0.1 mg/kg) do not impede Aβ₂₅₋₃₅-induced short-term memory deficits, enabling to set 0.1 mg/kg as a subactive dose for these two compounds (Table 1) and prepare the combinatorial study with the standard of care (Example 4)

### b. Step-Through Passive Avoidance Test (STPA)

After the Y-Maze test, the same animals also performed the step-through passive avoidance (STPA) test (eight and nine days after ICV injection of Aβ₂₅₋₃₅ peptide, D09 and D10 respectively), an index of contextual long-term memory. The apparatus consists in a two-compartment box (a lit compartment and a dark compartment) separated by a guillotine door. Foot shocks can be delivered into the dark compartment using a shock generator scrambler. On the first day (training session), each mouse is placed in the lit compartment with the guillotine door initially closed. After 5 sec, the door is raised. When the mouse enters the dark compartment and places all its paws on the grid floor, the door closes and a foot shock is delivered for 3 sec. Twenty-four (24) hours after this training session, the retention test (where no foot shock is performed) is carried out. Specifically, each mouse is placed again into the lit compartment with the door closed. After 5 sec, the door is raised. The time taken by the mouse to enter the dark compartment, called the step-through latency (STL), is recorded up to 300 sec. The time taken by the mouse to exit the dark compartment, called the escape latency (EL), is recorded up to 300 sec (Maurice et al., 1996, 1998; Meunier et al., 2006).

TABLE 2

| Groups | STL | | | | | |
|---|---|---|---|---|---|---|
| | Mean | SEM | % | n | Significance vs Aβ₂₅₋₃₅ / Vhc | p-value |
| Sc.Aβ / Vhc | 283.00 | 9.36 | 100 | 12 | *** | <0.001 |
| Aβ₂₅₋₃₅ / VhC | 122.58 | 6.73 | 43 | 12 | | |
| Aβ₂₅₋₃₅ / NX210 IP 0.1 | 118.92 | 7.24 | 42 | 12 | ns | >0.9999 |
| Aβ₂₅₋₃₅ / NX210 IP 1 | 177.92 | 9.66 | 63 | 12 | ns | >0.9999 |
| Aβ₂₅₋₃₅ / NX210 IP1/2d 2 | 263.33 | 13.72 | 93 | 12 | *** | <0.001 |
| Aβ₂₅₋₃₅ / NX210 IP 2 | 248.33 | 11.55 | 88 | 12 | *** | <0.001 |
| Aβ₂₅₋₃₅ / NX210 IP1/2d 3.75 | 264.75 | 9.89 | 94 | 12 | *** | <0.001 |
| Aβ₂₅₋₃₅ / NX210 IP 3.75 | 255.50 | 12.79 | 90 | 12 | *** | <0.001 |
| Aβ₂₅₋₃₅ / NX218 IP 0.1 | 129.75 | 9.26 | 46 | 12 | ns | >0.9999 |
| Aβ₂₅₋₃₅ / NX218 IP 1 | 252.42 | 13.49 | 89 | 12 | *** | <0.001 |
| Aβ₂₅₋₃₅ / NX218 IP1/2d 2 | 274.25 | 14.11 | 97 | 12 | *** | <0.001 |
| Aβ₂₅₋₃₅ / NX218 IP 2 | 267.75 | 12.41 | 95 | 12 | *** | <0.001 |
| Aβ₂₅₋₃₅ / NX218 IP 3.75 | 250.25 | 13.17 | 88 | 12 | *** | <0.001 |

Effect of IP administrations of NX210 or NX218 (0.1-1-2-3.75 mg/kg either once a day or every other day (1/2d)) on Aβ₂₅₋₃₅-induced long-term memory deficits in mice: step-through latency (STL) measured during the retention session. Doses are expressed in mg per kg. n is equal to 12 per group. Data are expressed in seconds. ns (non-significant), *** p < 0.001 vs. the Aβ₂₅₋₃₅ / Vhc group, Kruskal-Wallis followed by Dunn’s multiple comparison test

TABLE 3

| Groups | EL | | | | | |
|---|---|---|---|---|---|---|
| | Mean | SEM | % | n | Significance vs Aβ₂₅₋₃₅ / Vhc | p-value |
| Sc.Aβ / Vhc | 19.92 | 2.27 | 100 | 12 | *** | <0.001 |
| Aβ₂₅₋₃₅ / VhC | 89.50 | 5.51 | 449 | 12 | | |
| Aβ₂₅₋₃₅ / NX210 IP 0.1 | 89.42 | 6.16 | 449 | 12 | ns | >0.9999 |
| Aβ₂₅₋₃₅ / NX210 IP 1 | 53.08 | 4.58 | 267 | 12 | ns | >0.9999 |
| Aβ₂₅₋₃₅ / NX210 IP1/2d 2 | 22.00 | 2.76 | 110 | 12 | *** | <0.001 |
| Aβ₂₅₋₃₅ / NX210 IP 2 | 31.17 | 3.07 | 156 | 12 | ** | 0.0037 |
| Aβ₂₅₋₃₅ / NX210 IP1/2d 3.75 | 23.75 | 3.96 | 119 | 12 | *** | <0.001 |
| Aβ₂₅₋₃₅ / NX210 IP 3.75 | 27.67 | 2.86 | 139 | 12 | *** | <0.001 |
| Aβ₂₅₋₃₅ / NX218 IP 0.1 | 86.75 | 5.33 | 436 | 12 | ns | >0.9999 |
| Aβ₂₅₋₃₅ / NX218 IP 1 | 25.58 | 3.37 | 128 | 12 | *** | <0.001 |
| Aβ₂₅₋₃₅ / NX218 IP1/2d 2 | 24.83 | 3.62 | 125 | 12 | *** | <0.001 |
| Aβ₂₅₋₃₅ / NX218 IP 2 | 24.50 | 3.94 | 123 | 12 | *** | <0.001 |
| Aβ₂₅₋₃₅ / NX218 IP 3.75 | 26.50 | 3.83 | 133 | 12 | *** | <0.001 |

Effect of IP administrations of NX210 or NX218 (0.1-1-2-3.75 mg/kg either once a day or every other day (1/2d)) on Aβ₂₅₋₃₅-induced long-term memory deficits in mice: escape latency (EL) measured during the retention session. Doses are expressed in mg per kg. n is equal to 12 per group. Data are expressed in seconds. ns (non-significant), ** p<0.01 and *** p < 0.001 vs. the Aβ₂₅₋₃₅ / Vhc group, Kruskal-Wallis followed by Dunn’s multiple comparison test

The administrations once a day or every other day of NX210 (2 and 3.75 mg/kg) or NX218 (1, 2 and 3.75 mg/kg) fully rescued contextual long-term memory deficits (STL and EL), reaching levels similar to the non-injured Sham animals (Sc.Aβ / Vhc). This dose-effect demonstrates the strong efficacy of both NX210 and NX218 in counteracting cognitive deficits (long-term memory deficits) observed in this model of Tauopathies (Tables 2-3).

The administrations once a day of NX210 or NX218 (0.1 mg/kg) do not impede contextual long-term memory deficits (STL and EL), confirming that 0.1 mg/kg is a subactive dose for both compounds (Tables 2-3).

### c. Place Learning in the Morris Water-Maze - Reference Memory Test (MWM)

After the Y-Maze and instead of performing the STPA test, some animals performed the Morris Water-Maze (MWM) test in order to assess spatial working memory deficits (a six-day test from D09 to D14). This gold-standard test consists in a circular pool filled with opaque water placed in a room with external cues (sink, contrasted posters, shelves). A platform is immersed under the water surface during acquisition. Training consists in 3 swims per day for 5 days, performed between D09 to D13, with a 20 min inter-trial time. Each mouse is allowed to swim for 90 sec in order to find the platform, and to stay on it for 20 sec.

A probe test (PT) is performed 24 h after the last swim on D14. For the PT, the platform is removed, and each animal is allowed to swim for 60 sec. The start position for each mouse corresponds to one of two positions remote from the platform location in a counterbalanced order. The platform quadrant is named ‘target’ and other quadrants (opposite, adjacent right and adjacent left) are named ‘other’ during the PT. The time spent in each quadrant is recorded. The results are expressed as the percentage of time spent in the target quadrant compared to the mean percentage of time spent in the three other quadrants.

Every day, the latencies to find the platform during the first (swim1), second (swim2), third (swim3), fourth (swim4) and fifth (swim5) trials were monitored and averaged in this six-day experiment (Maurice et al., 2013).

TABLE 4

| Groups | MWM - Learning profile - Day 1 | | | | |
|---|---|---|---|---|---|
| | Mean | SEM | n | Significance vs Aβ₂₅₋₃₅ / Vhc | p-value |
| Sc.Aβ / Vhc | 82.73 | 2.34 | 11 | ns | 0.9797 |
| Aβ₂₅₋₃₅ / Vhc | 81.00 | 3.11 | 12 | | |
| Aβ₂₅₋₃₅ / NX210 IP 2 | 79.67 | 3.64 | 12 | ns | 0.9898 |
| Aβ₂₅₋₃₅ / NX218 IP 2 | 79.75 | 3.41 | 12 | ns | 0.9915 |
| Groups | MWM - Learning profile - Day 2 | | | | |
| | Mean | SEM | n | Significance vs Aβ₂₅₋₃₅ / Vhc | p-value |
| Sc.Aβ / Vhc | 54.27 | 2.54 | 11 | *** | <0.001 |
| Aβ₂₅₋₃₅ / Vhc | 75.33 | 3.43 | 12 | | |
| Aβ₂₅₋₃₅ / NX210 IP 2 | 53.58 | 3.96 | 12 | *** | <0.001 |
| Aβ₂₅₋₃₅ / NX218 IP 2 | 56.42 | 2.74 | 12 | *** | <0.001 |
| Groups | MWM - Learning profile - Day 3 | | | | |
| | Mean | SEM | n | Significance vs Aβ₂₅₋₃₅ / Vhc | p-value |
| Sc.Aβ / Vhc | 33.64 | 3.14 | 11 | *** | <0.001 |
| Aβ₂₅₋₃₅ / Vhc | 59.75 | 3.02 | 12 | | |
| Aβ₂₅₋₃₅ / NX210 IP 2 | 35.83 | 2.99 | 12 | *** | <0.001 |
| Aβ₂₅₋₃₅ / NX218 IP 2 | 31.42 | 2.61 | 12 | *** | <0.001 |
| Groups | MWM - Learning profile - Day 4 | | | | |
| | Mean | SEM | n | Significance vs Aβ₂₅₋₃₅ / Vhc | p-value |
| Sc.Aβ / Vhc | 26.91 | 2.62 | 11 | *** | <0.001 |
| Aβ₂₅₋₃₅ / Vhc | 56.50 | 4.41 | 12 | | |
| Aβ₂₅₋₃₅ / NX210 IP 2 | 25.92 | 1.98 | 12 | *** | <0.001 |
| Aβ₂₅₋₃₅ / NX218 IP 2 | 25.58 | 2.39 | 12 | *** | <0.001 |
| Sc.Aβ / Vhc | 21.09 | 1.93 | 11 | *** | <0.001 |
| Aβ₂₅₋₃₅ / Vhc | 55.08 | 4.19 | 12 | | |
| Aβ₂₅₋₃₅ / NX210 IP 2 | 24.25 | 2.52 | 12 | *** | <0.001 |
| Aβ₂₅₋₃₅ / NX218 IP 2 | 21.25 | 2.85 | 12 | *** | <0.001 |

Effect of IP administrations of NX210 or NX218 (2 mg/kg once a day) on Aβ₂₅₋₃₅-induced spatial working memory deficits in mice: time spent to reach the platform was determined during the five training days. Doses are expressed in mg per kg. n is equal to 11-12 per group. Data are expressed as the time spent to find the platform (in sec). ns (non-significant), *** p < 0.001 vs. the Aβ₂₅₋₃₅ / Vhc group, Two-way ANOVA followed by Tukey’s multiple comparison test

TABLE 5

| Groups | MWM - Probe test | | | | | |
|---|---|---|---|---|---|---|
| | | Mean | SEM | n | Significance vs Aβ₂₅₋₃₅ /Vhc | p-value |
| Sc.Aβ / Vhc | Target | 39.00 | 2.98 | 11 | *** | <0.001 |
| | Other | 20.50 | 0.98 | 11 | ns | 0.3925 |
| Aβ₂₅₋₃₅ / Vhc | Target | 21.56 | 3.46 | 12 | | |
| | Other | 26.31 | 1.15 | 12 | | |
| Aβ₂₅₋₃₅ / NX210 IP 2 | Target | 39.59 | 3.47 | 12 | *** | <0.001 |
| | Other | 20.30 | 1.15 | 12 | ns | 0.3415 |
| Aβ₂₅₋₃₅ / NX218 IP 2 | Target | 39.23 | 3.72 | 12 | *** | <0.001 |
| | Other | 20.49 | 1.24 | 12 | ns | 0.3701 |

Effect of IP administrations of NX210 or NX218 (2 mg/kg once a day) on Aβ₂₅₋₃₅-induced spatial working memory deficits in mice: time spent in each quadrant was determined during the probe test. Doses are expressed in mg per kg. n is equal to 11-12 per group. Data are expressed as the percentage of time spent in the target quadrant (Target) compared to the mean percentage of time spent in the three other quadrants (Other). ns (non-significant), *** p < 0.001 vs. the Aβ₂₅₋₃₅ / Vhc group, Two-way ANOVA followed by Tukey’s multiple comparison test

The administrations of either NX210 or NX218 significantly rescued the learning profile and the spatial long-term memory deficits in this model, as demonstrated by the Morris Water Maze results (Tables 4-5 and FIGS. 1-2). As soon as the second day of training (D10), mice treated with NX210 or NX218 were as efficient as the non-injured Sham mice (Sc.Aβ / Vhc) to remember the location of the hidden platform. The beneficial effects are kept all along the five-day training test (Table 4 and FIG. 1).

The beneficial effects on spatial memory were confirmed by the probe test (D14), a test where the platform was removed. In this test, NX210 or NX218-treated mice spent about 40% of total swim time in the target quadrant (i.e. the location of the removed platform) matching the non-injured Sham mice (Sc.Aβ / Vhc) in remembering the platform location. On the contrary, Aβ₂₅₋₃₅ / Vhc mice did not remember where the platform was and therefore spent roughly 25% of their swim time in each quadrant (Table 5 and FIG. 2).

This demonstrates once again the strong efficacy of both NX210 and NX218 in counteracting the cognitive deficits (spatial working memory deficits) observed in this model of Tauopathies.

Biochemical and histological assessments:

### a. Brain Biomarker Assessments

In order to evaluate potential biological effects of NX210 or NX218 treatments on pathological biomarkers, mice from the Sc.Aβ / Vhc (n=6), Aβ₂₅₋₃₅ / Vhc (n=6), Aβ₂₅₋₃₅ / NX210 IP 2 mg/kg (n=5) and Aβ₂₅₋₃₅ / NX218 IP 2 mg/kg (n=5) groups were sacrificed under anesthesia ten days after ICV injection of Aβ₂₅₋₃₅ peptide (D11) and daily compound administrations (once a day).

For each mouse, the brain was quickly removed and dissected out on an ice-cold metal plate into 2 hemi-hippocampi, 2 hemi-frontal cortices and the rest of brain. Straight after each collection, brain samples were frozen on dry ice and stored at -80° C. After thawing, brain structures were dissociated in 50 mM Tris-150 mM NaCl buffer, pH 7.5, and sonicated for 20 sec. After centrifugation, supernatants containing proteins were used for ELISA assays according to the manufacturer’s instructions (see below).
For Aβ₁₋₄₂ (amyloid-beta 1-42), use of the left hippocampus, ELISA kit from Cloud-Clone Corp., ref: CEA946Mu; batch: L190107385
For pTau (phosphorylated Tau protein), use of the left hippocampus, ELISA kit from Fisher Scientific, ref: 10591255; batch: 187860001

For all assays, the absorbance was read at 450 nm and each sample concentration was calculated by using a specific standard curve. All samples were run in duplicate and the average of these duplicates was used for calculations.

TABLE 6

| Groups | Aβ₁₋₄₂ | | | | | |
|---|---|---|---|---|---|---|
| | Mean | SEM | % | n | Significance vs Aβ₂₅₋₃₅ / Vhc | p-value |
| Sc.Aβ / Vhc | 22.63 | 6.91 | 100 | 6 | *** | <0.001 |
| Aβ₂₅₋₃₅ / VhC | 59.24 | 4.12 | 262 | 6 | | |
| Aβ₂₅₋₃₅ / NX210 IP 2 | 24.25 | 2.93 | 107 | 5 | *** | <0.001 |
| Aβ₂₅₋₃₅ / NX218 IP 2 | 23.90 | 0.98 | 106 | 5 | *** | <0.001 |

| Groups | pTau | | | | | |
|---|---|---|---|---|---|---|
| | Mean | SEM | % | n | Significance vs Aβ₂₅₋₃₅ / Vhc | p-value |
| Sc.Aβ / Vhc | 59.82 | 1.95 | 100 | 6 | *** | <0.001 |
| Aβ₂₅₋₃₅ / VhC | 112.73 | 1.76 | 188 | 6 | | |
| Aβ₂₅₋₃₅ / NX210 IP 2 | 90.21 | 1.69 | 151 | 5 | *** | <0.001 |
| Aβ₂₅₋₃₅ / NX218 IP 2 | 60.41 | 1.19 | 101 | 5 | *** | <0.001 |

Effect of IP administrations of NX210 or NX218 (2 mg/kg once a day) on Aβ₂₅₋₃₅-induced biochemical changes in brain mice (Aβ₁₋₄₂ and phosphorylated Tau levels). Doses are expressed in mg per kg. n is equal to 5-6 per group. Data are expressed as a percentage of the control group (Sc.Aβ / Vhc group). ns (non-significant), *** p < 0.001 vs. the Aβ₂₅₋₃₅ /Vhc group, One- Way ANOVA followed by Dunnett’s test

In this model of Tauopathies, when compared to Aβ₂₅₋₃₅ / Vhc group the administrations once a day for ten days of NX210 (2 mg/kg) or NX218 (2 mg/kg) surprisingly and significantly decreased several brain biomarkers typically involved in the progression of AD and other Tauopathies such as Amyloid Beta 1-42 (Aβ₁₋₄₂) and hyperphosphorylated Tau protein (pTau) (Table 6 and FIG. 3). In fact, the levels of Aβ₁₋₄₂ and pTau were similar to those of healthy Sham animals (for both NX210 and NX218-treated groups, and for NX218-treated group respectively).

### b. Histological Study

In order to measure neuronal loss in this model (as described by Maurice et al., 2013), a well-known pathological event occurring in patients with AD/Tauopathies, hippocampal neurons were counted in the brain of Sham, Aβ₂₅₋₃₅ / vehicle and Aβ₂₅₋₃₅ / NX210-treated mice.

To do so, fourteen days after ICV injection of Aβ₂₅₋₃₅ or Scrambled peptide (D15) and daily compound administrations of NX210 (IP) or vehicle (IP), mice were anaesthetized by ketamine/xylazine and then transcardially perfused with phosphate buffer solution (PBS, 15 ml at 5 ml/min, pH 7.4) until fluid running clear, followed by a solution of 4% paraformaldehyde (PFA, 25 ml at 5 ml/min) until the fixation tremors disappeared. Brains were removed carefully and kept in 4% PFA at 4° C. for 24-48 h. Brains were then dehydrated through a series of graded ethanol baths to displace the water, and then infiltrated with wax. Finally, the infiltrated brains were embedded into wax blocks and sectioned at 5±1 pm. Cresyl violet (CV) staining was performed on 9 brain sections per animal, each coronal section separated by 100 µm for CA1 pyramidal neuron counting.

TABLE 7

| Groups | Histology (number of neurons per mm of tissue) | | | | |
|---|---|---|---|---|---|
| | Mean | SEM | % | Significance vs Aβ₂₅₋₃₅ / Vhc | p-value |
| Sc.Aβ / Vhc | 335.49 | 8.20 | 100 | *** | <0.001 |
| Aβ₂₅₋₃₅ / Vhc | 276.64 | 9.84 | 82 | | |
| Aβ₂₅₋₃₅ / NX210 IP 2 | 311.84 | 9.18 | 93 | * | 0.0192 |

Effect of IP administrations of NX210 (2 mg/kg once a day) on neuronal loss observed in Aβ₂₅₋₃₅-injected mice. Data are expressed as a ratio of the number of nucleus over the length of the analyzed area and as a percentage of control (Sc.Aβ / Vhc). * p < 0.05 and ***p < 0.001 vs. the Aβ₂₅₋₃₅ / Vhc group, One-Way ANOVA followed by Dunnett’s test

NX210 (2 mg/kg) significantly prevents the neuronal loss observed in the hippocampus of mice injected with Aβ₂₅₋₃₅ peptide (Table 7). Only eighty-two (82%) percent of pyramidal neurons were remaining in the Aβ₂₅₋₃₅ / Vhc mice (versus Sham mice) in comparison to ninety-three (93%) percent after treatment with NX210.

### Example 4: Combination Standard of Care With NX210 or NX218

Acetylcholine esterase inhibitors such as Donepezil (DPZ) represent the current standard of care for patients suffering from Alzheimer’s disease or Tauopathies. The efficacy response to these compounds is unpredictable, and with a loss of efficacy seen over time. Increasing the dose is only a temporary option as the side effects associated with this increased dosage are usually not well tolerated by the patients (Homma et al., 2009; Jackson et al., 2004). Ultimately, patients will therefore stop their treatment, leaving them without a single therapeutic solution.

### Cognitive Assessments

### a. Spontaneous Alternation Performance (Y-Maze)

Seven days after ICV injection of Aβ₂₅₋₃₅ peptide (D08), all animals were tested for spontaneous alternation performance in the Y-maze, an index of spatial working memory (short-term memory test, see Example 3 for methodological details).

TABLE 8

| Groups | Y-maze | | | | | |
|---|---|---|---|---|---|---|
| | Mean | SEM | % | n | Significance vs Aβ₂₅₋₃₅ / Vhc | p-value |
| Sc.Aβ / Vhc | 74.04 | 1.00 | 100 | 12 | *** | <0.001 |
| Aβ₂₅₋₃₅ / VhC | 38.95 | 2.34 | 53 | 12 | | |
| Aβ₂₅₋₃₅ / NX210 IP 0.1 | 44.52 | 1.70 | 60 | 12 | ns | 0.3097 |
| Aβ₂₅₋₃₅ / NX210 IP 2 | 72.67 | 1.48 | 98 | 12 | *** | <0.001 |
| Aβ₂₅₋₃₅ / NX218 IP 0.1 | 43.27 | 2.66 | 58 | 12 | ns | 0.5957 |
| Aβ₂₅₋₃₅ / NX218 IP 2 | 72.53 | 2.51 | 98 | 12 | *** | <0.001 |
| Aβ₂₅₋₃₅ / DPZ PO 0.25 | 41.82 | 3.16 | 56 | 12 | ns | 0.9172 |
| Aβ₂₅₋₃₅ / DPZ PO 1 | 68.90 | 2.19 | 93 | 12 | *** | <0.001 |
| Aβ₂₅₋₃₅ / DPZ 0.25 + NX210 0.1 | 67.88 | 1.30 | 92 | 12 | *** | <0.001 |
| Aβ₂₅₋₃₅ / DPZ 0.25 + NX218 0.1 | 71.94 | 1.32 | 97 | 12 | *** | <0.001 |

Effect of administrations of active or subactive doses of NX210 or NX218 (0.1-2 mg/kg once a day, IP), or of DPZ (0.25-1 mg/kg once a day, PO) or combination of subactive doses of NX210 or NX218 with subactive doses of DPZ (0.1 mg/kg for NX210 and NX218, IP, and 0.25 mg/kg for DPZ, PO) on Aβ₂₅₋₃₅-induced short-term memory deficits in mice. Doses are expressed in mg per kg. n is equal to 12 per group. Data are expressed as a percentage of alternation. ns (non-significant), ***p < 0.001 vs. the Aβ₂₅₋₃₅ / Vhc group, One-Way ANOVA followed by Dunnett’s test

At subactive doses, the administrations once a day of NX210 (0.1 mg/kg), NX218 (0.1 mg/kg) or DPZ (0.25 mg/kg) do not impede Aβ₂₅₋₃₅-induced spatial short-term working memory deficits (Table 8 and FIG. 4, see also Example 3).

Surprisingly, we demonstrate here that the combination of NX210 or of NX218 at a subactive dose (0.1 mg/kg, IP) with DPZ at a subactive dose (0.25 mg/kg, PO) significantly and fully restores Aβ₂₅₋₃₅-induced spatial short-term working memory deficits (Table 8 and FIG. 4), showing a synergistic therapeutic effect between the drugs on Tauopathies-related cognitive impairments.

### b. Step-Through Passive Avoidance Test (STPA)

Eight and nine days after ICV injection of Aβ₂₅₋₃₅ peptide (D09 and D10 respectively), the animals were tested for contextual long-term memory performance in the step-through passive avoidance (STPA) test (see Example 3 for method details).

TABLE 9

| Groups | STL | | | | | |
|---|---|---|---|---|---|---|
| | Mean | SEM | % | n | Significance vs Aβ₂₅₋₃₅ / Vhc | p-value |
| Sc.Aβ / Vhc | 283.00 | 9.36 | 100 | 12 | *** | <0.001 |
| Aβ₂₅₋₃₅ / VhC | 122.58 | 6.73 | 43 | 12 | | |
| Aβ₂₅₋₃₅ / NX210 IP 0.1 | 118.92 | 7.24 | 42 | 12 | ns | >0.9999 |
| Aβ₂₅₋₃₅ / NX210 IP 2 | 248.33 | 11.55 | 88 | 12 | ** | 0.0018 |
| Aβ₂₅₋₃₅ / NX218 IP 0.1 | 129.75 | 9.26 | 46 | 12 | ns | >0.9999 |
| Aβ₂₅₋₃₅ / NX218 IP 2 | 267.75 | 12.41 | 95 | 12 | *** | <0.001 |
| Aβ₂₅₋₃₅ / DPZ PO 0.25 | 109.33 | 10.50 | 39 | 12 | ns | >0.9999 |
| Aβ₂₅₋₃₅ / DPZ PO 1 | 265.83 | 11.65 | 94 | 12 | *** | <0.001 |
| Aβ₂₅₋₃₅ / DPZ 0.25 + NX210 0.1 | 264.25 | 12.43 | 93 | 12 | *** | <0.001 |
| Aβ₂₅₋₃₅ / DPZ 0.25 + NX218 0.1 | 263.42 | 12.58 | 93 | 12 | *** | <0.001 |

Effect of administrations of active or subactive doses of NX210 or NX218 (0.1-2 mg/kg once a day, IP), or of DPZ (0.25-1 mg/kg once a day, PO) or combination of subactive doses of NX210 or NX218 with subactive doses of DPZ (0.1 mg/kg for NX210 and NX218, IP, and 0.25 mg/kg for DPZ, PO) on Aβ₂₅₋₃₅-induced long-term memory deficits in mice : step-through latency (STL) measured during the retention session. Doses are expressed in mg per kg. n is equal to 12 per group. Data are expressed in seconds, ns (non-significant), **p<0.01 and *** p < 0.001 vs. the Aβ₂₅₋₃₅ / Vhc group, Kruskal-Wallis followed by Dunn’s multiple comparison test

TABLE 10

| Groups | EL | | | | | |
|---|---|---|---|---|---|---|
| | Mean | SEM | % | n | Significance vs Aβ₂₅₋₃₅ / Vhc | p-value |
| Sc.Aβ / Vhc | 19.92 | 2.27 | 100 | 12 | *** | <0.001 |
| Aβ₂₅₋₃₅ / Vhc | 89.50 | 5.51 | 449 | 12 | | |
| Aβ₂₅₋₃₅ / NX210 IP 0.1 | 89.42 | 6.16 | 449 | 12 | ns | >0.9999 |
| Aβ₂₅₋₃₅ / NX210 IP 2 | 31.17 | 3.07 | 156 | 12 | * | 0.0106 |
| Aβ₂₅₋₃₅ / NX218 IP 0.1 | 86.75 | 5.33 | 436 | 12 | ns | >0.9999 |
| Aβ₂₅₋₃₅ / NX218 IP 2 | 24.50 | 3.94 | 123 | 12 | *** | <0.001 |
| Aβ₂₅₋₃₅ / DPZ PO 0.25 | 88.67 | 6.67 | 445 | 12 | ns | >0.9999 |
| Aβ₂₅₋₃₅ / DPZ PO 1 | 24.25 | 2.84 | 122 | 12 | *** | <0.001 |
| Aβ₂₅₋₃₅ / DPZ 0.25 + NX210 0.1 | 19.50 | 3.08 | 98 | 12 | *** | <0.001 |
| Aβ₂₅₋₃₅ / DPZ 0.25 + NX218 0.1 | 19.17 | 3.04 | 96 | 12 | *** | <0.001 |

Effect of administrations of active or subactive doses of NX210 or NX218 (0.1-2 mg/kg once a day, IP), or of DPZ (0.25-1 mg/kg once a day, PO) or combination of subactive doses of NX210 or NX218 with subactive doses of DPZ (0.1 mg/kg for NX210 and NX218, IP, and 0.25 mg/kg for DPZ, PO) on Aβ₂₅₋₃₅-induced long-term memory deficits in mice: escape latency (EL) measured during the retention session. Doses are expressed in mg per kg. n is equal to 12 per group. Data are expressed in seconds, ns (non-significant), * p < 0.05 and ***p < 0.001 vs. the Aβ₂₅₋₃₅ / Vhc group, Kruskal-Wallis followed by Dunn’s multiple comparison test

At subactive doses, the administrations once a day of NX210 (0.1 mg/kg), NX218 (0.1 mg/kg) or DPZ (0.25 mg/kg) do not impede Aβ₂₅₋₃₅-induced contextual long-term memory deficits (STL and EL, Tables 9-10 and FIG. 5, see also Example 3).

Surprisingly, we demonstrate in this test that the combination of NX210 or NX218 at their subactive dose (0.1 mg/kg, IP) with DPZ at its subactive dose (0.25 mg/kg, PO) significantly and fully restores Aβ₂₅₋₃₅-induced long-term memory deficits (STL and EL, Tables 9-10 and FIG. 5). This result confirms a synergistic beneficial effect of the combination of NX210 or NX218 with an inhibitor of acetylcholine esterase (here Donepezil) on Tauopathies-related cognitive impairments.

### Example 5: Late Stage Efficacy of Nx210 or Nx218

### Cognitive Assessments

### a. Spontaneous Alternation Performance (Y-Maze)

In the Aβ₂₅₋₃₅ mouse model of Tauopathies, ten days after ICV injection of Aβ₂₅₋₃₅ peptide (D11) the physiopathology is already at a very late-stage as demonstrated by the high cognitive impairments seen in the Y-maze and STPA tests (Example 3 Tables 1-2-3, vehicle groups) as well as by the strong alterations of the two pathological brain biomarkers Amyloid Beta 1-42 (Aβ₁₋₄₂) and hyperphosphorylated Tau protein (pTau) (Example 3 Table 6 and FIG. 3, vehicle groups).

The efficacy of NX210 and NX218 SCO-Spondin derived peptides was therefore assessed during this late-stage physiopathology setting. Specifically, the administrations of NX210, NX218 or vehicle were started at D11 and were repeated once a day until the end of the experiment.

All animals were tested for spontaneous alternation performance in the Y-maze, an index of short-term memory (see Example 3 for method details) from D08 (i.e. pre-treatment values) and then once a week for three weeks (D15, D22, D29).

TABLE 11

| Groups | Y-maze (D08) | | | | | |
|---|---|---|---|---|---|---|
| | Mean | SEM | % | n | Significance vs Aβ₂₅₋₃₅ / D11 vhc | p-value |
| Sc.Aβ / D11 vhc | 70.68 | 2.70 | 100 | 6 | *** | <0.001 |
| Aβ₂₅₋₃₅ / D11 vhc | 40.57 | 1.83 | 57 | 6 | | |
| Aβ₂₅₋₃₅ / D11 NX210 IP 2 | 39.17 | 2.17 | 55 | 6 | ns | 0.9469 |
| Aβ₂₅₋₃₅ / D11 NX218 IP 2 | 40.70 | 2.27 | 58 | 6 | ns | >0.9999 |

| Groups | Y-maze (D15) | | | | | |
|---|---|---|---|---|---|---|
| | Mean | SEM | % | n | Significance vs Aβ₂₅₋₃₅ / D11 vhc | p-value |
| Sc.Aβ / D11 vhc | 78.47 | 2.57 | 100 | 6 | *** | <0.001 |
| Aβ₂₅₋₃₅ / D11 vhc | 47.40 | 3.23 | 60 | 6 | | |
| Aβ₂₅₋₃₅ / D11 NX210 IP 2 | 55.55 | 1.51 | 71 | 6 | ns | 0.1887 |
| Aβ₂₅₋₃₅ / D11 NX218 IP 2 | 57.32 | 4.44 | 73 | 6 | ns | 0.0904 |
| Groups | Y-maze (D22) | | | | | |
| | Mean | SEM | % | n | Significance vs Aβ₂₅₋₃₅ / D11 vhc | p-value |
| Sc.Aβ / D11 vhc | 73.42 | 2.23 | 100 | 6 | *** | <0.001 |
| Aβ₂₅₋₃₅ / D11 vhc | 40.70 | 2.17 | 55 | 6 | | |
| Aβ₂₅₋₃₅ / D11 NX210 IP 2 | 60.08 | 2.26 | 82 | 6 | *** | <0.001 |
| Aβ₂₅₋₃₅ / D11 NX218 IP 2 | 60.37 | 2.47 | 82 | 6 | *** | <0.001 |
| Groups | Y-maze (D29) | | | | | |
| | Mean | SEM | % | n | Significance vs Aβ₂₅₋₃₅ / D11 vhc | p-value |
| Sc.Aβ / D11 Vhc | 77.32 | 1.52 | 100 | 6 | *** | <0.001 |
| Aβ₂₅₋₃₅ / D11 Vhc | 47.82 | 3.59 | 62 | 6 | | |
| Aβ₂₅₋₃₅ / D11 NX210 IP 2 | 74.73 | 2.64 | 97 | 6 | *** | <0.001 |
| Aβ₂₅₋₃₅ / D11 NX218 IP 2 | 70.10 | 3.70 | 91 | 6 | *** | <0.001 |

Effect of administrations of NX210 or NX218 (2 mg/kg once a day started at D11) on Aβ₂₅₋₃₅-induced short-term memory deficits in mice. Doses are expressed in mg per kg. n is equal to 6 per group. Data are expressed as a percentage of alternation over time (D08-15-22-29). ns (non-significant), *** p < 0.001 vs. the Aβ₂₅₋₃₅ / Vhc group, One-Way ANOVA followed by Dunnett’s test

At D08 (pre-treatment value), strong memory impairments were similarly observed in all Aβ₂₅₋₃₅ groups when compared to Sc.Aβ / D11 vhc (Sham) group (Table 11 and FIG. 6).

At D15, five daily administrations of NX210 or NX218 (2 mg/kg) already demonstrated a tendency to rescue Aβ₂₅₋₃₅-induced spatial short-term working memory deficits (p-value = 0.19 and 0.09 for NX210 and NX218 respectively, Table 11 and FIG. 6). At D22 after a 12 day-treatment, the administrations of NX210 or NX218 significantly rescued working memory deficits (Table 11 and FIG. 6). Finally, at D29 after a 19 day-treatment the administrations of NX210 or NX218 fully restored the memory deficits observed in this Tauopathy model (97% and 91% compared to Sham group respectively, Table 11 and FIG. 6).

In conclusion, NX210 and NX218 were able to fully restore the cognitive and memory deficits of this model of Tauopathies, even with a treatment started at a very advanced pathophysiological stage (late disease-onset).

### Example 6: Long-Term Effects of NX218 and/or DPZ Based Treatments

To investigate the potential of NX218 treatment to maintain cognitive restoration for a longer duration and to work as a 2^{nd} line treatment when DPZ lose its activity: a long-term study (120 days) was performed in the Aβ₂₅₋₃₅ mouse model of Tauopathies.

Mice were assigned to 6 treatment groups with 5-6 animals per group as described below.

| Group # | Group treatments | n |
|---|---|---|
| Group 1 | Sc.Aβ + vehicle IP o.d. starting D01 until D120 | 5 |
| Group 2 | Aβ₂₅₋₃₅ + vehicle IP o.d. starting D01 until D120 | 5 |
| Group 3 | Aβ₂₅₋₃₅ + test compound NX218 IP o.d. starting D11 until D38 (2 mg/kg) | 6 |
| Group 4 | Aβ₂₅₋₃₅ + test compound NX218 IP o.d. starting D01 until D120 (2 mg/kg) | 6 |
| Group 5 | Aβ₂₅₋₃₅ + DPZ PO o.d. (1 mg/kg) starting D01 until not effective anymore (D43), then treated with NX218 IP (2 mg/kg) from D44 until D78, with NX218 IP (4 mg/kg) from D79 until D99, with NX218 IP (8 mg/kg) from D100 until D113, and washout from D114 until D120 | 6 |
| Group 6 | Aβ₂₅₋₃₅ + NX218 IP (0.1 mg/kg) + DPZ PO (0.25 mg/kg) o.d. starting D01 until D120 | 6 |
| | Total mice | 34 |

Sc.Aβ or Aβ₂₅₋₃₅ peptides were injected ICV to provoke (Aβ₂₅₋₃₅) or not (Sc.Aβ) amyloid toxicity on day 1 (D01).

### Control Groups

From D01 until D120, mice of groups 1 and 2 were administered IP once a day (o.d.) with vehicle (water for injection).

Test compound NX218:
From D11 until D38, mice of group 3 were administered IP o.d. with NX218 at 2 mg/kg (mice were not treated with vehicle from D01 until D10).
From D01 until D120, mice of group 4 were administered IP o.d. with NX218 at 2 mg/kg.

DPZ rescued by test compound NX218:
From D01, mice of group 5 were administered PO by gavage o.d with DPZ at its active dose (1 mg/kg) until its effect disappears. Its efficacy disappeared on D36. DPZ was administered until D43 and then from D44, NX218 replaced DPZ until D78 via IP administration once a day at its active dose (2 mg/kg).
From D79 until D99: NX218 was administered via IP administration o.d. at a higher dose (4 mg/kg).
From D100 until D113 NX218 was administered via IP administration o.d. at a higher dose (8 mg/kg).
Administration of NX218 was stopped from D114 until animals were sacrificed.

Test compound NX218 in combination with DPZ:
From D01 until D120, mice of group 6 were co-administered with NX218 and DPZ at their respective subactive doses:
   NX218: IP o.d., 0.1 mg/kg
   DPZ: PO o.d., 0.25 mg/kg

### Cognitive Assessments

All groups performed one behavioral test every week starting on D08 (7 days after Aβ₂₅₋₃₅ peptide injection) to monitor the effects of the compounds:

Spontaneous alternation procedure in the Y-maze (YM, assessing spatial short-term/working memory as described in example 3) on D08, D15, D22, D29, D36, D43, D50, D57, D64, D71, D78, D85, D92, D99, D106, D113 and D120. Results are presented in Table 12 and FIG. 7.

TABLE 12

| Groups | Y-maze (D08) | | | | | |
|---|---|---|---|---|---|---|
| | Mean | SEM | % | n | Significance vs Aβ₂₅₋₃₅ / vhc IP D01-D120 | p-value |
| Group 1 | 73.88 | 1.11 | 100 | 5 | *** | <0.001 |
| Group 2 | 36.72 | 2.82 | 50 | 5 | | |
| Group 3 | 45.65 | 3.24 | 62 | 6 | ns | 0.2679 |
| Group 4 | 71.18 | 1.14 | 96 | 6 | *** | <0.001 |
| Group 5 | 72.08 | 2.72 | 98 | 6 | *** | <0.001 |
| Group 6 | 71.28 | 3.12 | 96 | 6 | *** | <0.001 |
| Groups | Y-maze (D15) | | | | | |
| | Mean | SEM | % | n | Significance vs Aβ₂₅₋₃₅ / vhc IP D01-D120 | p-value |
| Group 1 | 77.9 | 2.26 | 100 | 5 | *** | <0.001 |
| Group 2 | 46.00 | 2.23 | 59 | 5 | | |
| Group 3 | 56.48 | 1.55 | 73 | 6 | ns | 0.0947 |
| Group 4 | 78.42 | 1.73 | 101 | 6 | *** | <0.001 |
| Group 5 | 76.00 | 1.83 | 98 | 6 | *** | <0.001 |
| Group 6 | 72.83 | 3.00 | 93 | 6 | *** | <0.001 |
| Groups | Y-maze (D22) | | | | | |
| | Mean | SEM | % | n | Significance vs Aβ₂₅₋₃₅ / vhc IP D01-D120 | p-value |
| Group 1 | 74.42 | 1.14 | 100 | 5 | *** | <0.001 |
| Group 2 | 48.88 | 2.43 | 66 | 5 | | |
| Group 3 | 64.83 | 2.56 | 87 | 6 | *** | <0.001 |
| Group 4 | 73.88 | 1.98 | 99 | 6 | *** | <0.001 |
| Group 5 | 79.07 | 1.48 | 106 | 6 | *** | <0.001 |
| Group 6 | 72.48 | 2.27 | 97 | 6 | *** | <0.001 |
| Groups | Y-maze (D29) | | | | | |
| | Mean | SEM | % | n | Significance vs Aβ₂₅₋₃₅ / vhc IP D01-D120 | p-value |
| Group 1 | 77.44 | 1.49 | 100 | 5 | *** | <0.001 |
| Group 2 | 36.3 | 2.75 | 47 | 5 | | |
| Group 3 | 76.38 | 2.64 | 99 | 6 | *** | <0.001 |
| Group 4 | 72.17 | 1.83 | 93 | 6 | *** | <0.001 |
| Group 5 | 62.05 | 3.57 | 80 | 6 | *** | <0.001 |
| Group 6 | 75.77 | 1.98 | 98 | 6 | *** | <0.001 |
| Groups | Y-maze (D36) | | | | | |
| | Mean | SEM | % | n | Significance vs Aβ₂₅₋₃₅ / vhc IP D01-D120 | p-value |
| Group 1 | 74.42 | 4.26 | 100 | 5 | *** | <0.001 |
| Group 2 | 42.58 | 2.20 | 57 | 5 | | |
| Group 3 | 77.42 | 2.74 | 104 | 6 | *** | <0.001 |
| Group 4 | 75.48 | 1.96 | 101 | 6 | *** | <0.001 |
| Group 5 | 36.23 | 1.86 | 49 | 6 | ns | 0.7918 |
| Group 6 | 75.4 | 2.09 | 101 | 6 | *** | <0.001 |
| Groups | Y-maze (D43) | | | | | |
| | Mean | SEM | % | n | Significance vs Aβ₂₅₋₃₅ / vhc IP D01-D120 | p-value |
| Group 1 | 74.16 | 2.28 | 100 | 5 | *** | <0.001 |
| Group 2 | 35.92 | 2.55 | 48 | 5 | | |
| Group 3 | 69.88 | 2.67 | 94 | 6 | *** | <0.001 |
| Group 4 | 73.2 | 2.49 | 99 | 6 | *** | <0.001 |
| Group 5 | 38.73 | 4.15 | 52 | 6 | ns | >0.999 |
| Group 6 | 73.5 | 1.38 | 99 | 6 | *** | <0.001 |
| Groups | Y-maze (D50) | | | | | |
| | Mean | SEM | % | n | Significance vs Aβ₂₅₋₃₅ / vhc IP D01-D120 | p-value |
| Group 1 | 73.5 | 1.28 | 100 | 5 | *** | <0.001 |
| Group 2 | 40.08 | 1.88 | 55 | 5 | | |
| Group 3 | 76.6 | 1.70 | 104 | 6 | *** | <0.001 |
| Group 4 | 76.9 | 0.78 | 105 | 6 | *** | <0.001 |
| Group 5 | 55.08 | 3.69 | 75 | 6 | ** | 0.002 |
| Group 6 | 77.85 | 1.08 | 106 | 6 | *** | <0.001 |
| Groups | Y-maze (D57) | | | | | |
| | Mean | SEM | % | n | Significance vs Aβ₂₅₋₃₅ / vhc IP D01-D120 | p-value |
| Group 1 | 77.2 | 7.77 | 100 | 5 | *** | <0.001 |
| Group 2 | 42.94 | 2.31 | 56 | 5 | | |
| Group 3 | 76.38 | 2.48 | 99 | 6 | *** | <0.001 |
| Group 4 | 69.1 | 2.13 | 90 | 6 | *** | <0.001 |
| Group 5 | 67.87 | 1.62 | 88 | 6 | *** | <0.001 |
| Group 6 | 72.25 | 2.60 | 94 | 6 | *** | <0.001 |
| Groups | Y-maze (D64) | | | | | |
| | Mean | SEM | % | n | Significance vs Aβ₂₅₋₃₅ / vhc IP D01-D120 | p-value |
| Group 1 | 75.1 | 3.36 | 100 | 5 | *** | <0.001 |
| Group 2 | 45.58 | 3.53 | 61 | 5 | | |
| Group 3 | 77.97 | 1.61 | 104 | 6 | *** | <0.001 |
| Group 4 | 70.92 | 3.13 | 94 | 6 | *** | <0.001 |
| Group 5 | 77.07 | 6.00 | 103 | 6 | *** | <0.001 |
| Group 6 | 73.95 | 2.79 | 98 | 6 | *** | <0.001 |
| Groups | Y-maze (D71) | | | | | |
| | Mean | SEM | % | n | Significance vs Aβ₂₅₋₃₅ / vhc IP D01-D120 | p-value |
| Group 1 | 74.94 | 3.24 | 100 | 5 | *** | <0.001 |
| Group 2 | 35.06 | 3.75 | 47 | 5 | | |
| Group 3 | 74.52 | 3.46 | 99 | 6 | *** | <0.001 |
| Group 4 | 75.23 | 2.65 | 100 | 6 | *** | <0.001 |
| Group 5 | 61.6 | 4.06 | 82 | 6 | *** | <0.001 |
| Group 6 | 78.05 | 1.73 | 104 | 6 | *** | <0.001 |
| Groups | Y-maze (D78) | | | | | |
| | Mean | SEM | % | n | Significance vs Aβ₂₅₋₃₅ / vhc IP D01-D120 | p-value |
| Group 1 | 79.54 | 1.53 | 100 | 5 | *** | <0.001 |
| Group 2 | 35.4 | 3.10 | 45 | 5 | | |
| Group 3 | 75.77 | 1.62 | 95 | 6 | *** | <0.001 |
| Group 4 | 77.82 | 1.68 | 98 | 6 | *** | <0.001 |
| Group 5 | 53.25 | 3.55 | 67 | 6 | *** | <0.001 |
| Group 6 | 75.53 | 1.61 | 95 | 6 | *** | <0.001 |
| Groups | Y-maze (D85) | | | | | |
| | Mean | SEM | % | n | Significance vs Aβ₂₅₋₃₅ / vhc IP D01-D120 | p-value |
| Group 1 | 78.24 | 2.37 | 100 | 5 | *** | <0.001 |
| Group 2 | 45.92 | 4.94 | 59 | 5 | | |
| Group 3 | 76.07 | 2.21 | 97 | 6 | *** | <0.001 |
| Group 4 | 76.12 | 1.81 | 97 | 6 | *** | <0.001 |
| Group 5 | 56.77 | 2.63 | 73 | 6 | ns | 0.072 |
| Group 6 | 77.55 | 3.42 | 99 | 6 | *** | <0.001 |
| Groups | Y-maze (D92) | | | | | |
| | Mean | SEM | % | n | Significance vs Aβ₂₅₋₃₅ / vhc IP D01-D120 | p-value |
| Group 1 | 78.3 | 1.82 | 100 | 5 | *** | <0.001 |
| Group 2 | 41.28 | 3.95 | 53 | 5 | | |
| Group 3 | 74.08 | 2.79 | 95 | 6 | *** | <0.001 |
| Group 4 | 76.28 | 2.72 | 97 | 6 | *** | <0.001 |
| Group 5 | 63.35 | 2.80 | 81 | 6 | *** | <0.001 |
| Group 6 | 71.38 | 4.58 | 91 | 6 | *** | <0.001 |
| Groups | Y-maze (D99) | | | | | |
| | Mean | SEM | % | n | Significance vs Aβ₂₅₋₃₅ / vhc IP D01-D120 | p-value |
| Group 1 | 77.42 | 2.13 | 100 | 5 | *** | <0.001 |
| Group 2 | 41.28 | 2.89 | 53 | 5 | | |
| Group 3 | 78.35 | 1.43 | 101 | 6 | *** | <0.001 |
| Group 4 | 77.03 | 2.58 | 100 | 6 | *** | <0.001 |
| Group 5 | 60.47 | 4.66 | 78 | 6 | *** | <0.001 |
| Group 6 | 77.55 | 1.75 | 100 | 6 | *** | <0.001 |
| Groups | Y-maze (D106) | | | | | |
| | Mean | SEM | % | n | Significance vs Aβ₂₅₋₃₅ / vhc IP D01-D120 | p-value |
| Group 1 | 75.82 | 1.69 | 100 | 5 | *** | <0.001 |
| Group 2 | 36.38 | 3.15 | 48 | 5 | | |
| Group 3 | 73.98 | 3.09 | 98 | 6 | *** | <0.001 |
| Group 4 | 73.2 | 1.58 | 97 | 6 | *** | <0.001 |
| Group 5 | 68.93 | 2.15 | 91 | 6 | *** | <0.001 |
| Group 6 | 73.5 | 1.31 | 97 | 6 | *** | <0.001 |
| Groups | Y-maze (D113) | | | | | |
| | Mean | SEM | % | n | Significance vs Aβ₂₅₋₃₅ / vhc IP D01-D120 | p-value |
| Group 1 | 76.44 | 3.47 | 100 | 5 | *** | <0.001 |
| Group 2 | 48.48 | 4.48 | 63 | 5 | | |
| Group 3 | 74.33 | 2.21 | 97 | 6 | *** | <0.001 |
| Group 4 | 81.03 | 2.39 | 106 | 6 | *** | <0.001 |
| Group 5 | 77.43 | 1.94 | 101 | 6 | *** | <0.001 |
| Group 6 | 75.62 | 2.00 | 99 | 6 | *** | <0.001 |
| Groups | Y-maze (D120) | | | | | |
| | Mean | SEM | % | n | Significance vs Aβ₂₅₋₃₅ / vhc IP D01-D120 | p-value |
| Group 1 | 77.24 | 1.72 | 100 | 5 | *** | <0.001 |
| Group 2 | 45.86 | 3.26 | 59 | 5 | | |
| Group 3 | 76.62 | 2.64 | 99 | 6 | *** | <0.001 |
| Group 4 | 76.28 | 2.80 | 99 | 6 | *** | <0.001 |
| Group 5 | 79.75 | 2.39 | 103 | 6 | *** | <0.001 |
| Group 6 | 79.47 | 1.06 | 103 | 6 | *** | <0.001 |

Long-term effect of administrations of NX218 (2 mg/kg IP once a day for 120 days (group 4)) or DPZ (1 mg/kg per os once a day for 43 days) replaced by increasing doses of NX218 (2 mg/kg IP once a day from day 44 to day 78, then 4 mg/kg once a day from day 79 to day 99 and then 8 mg/kg once a day from day 100 to day 113 (group 5)) or subactivedoses of NX218 (0.1 mg/kg IP once a day for 120 days) and DPZ (0.25 mg/kg per os once a day for 120 days) combined (group 6) or transient treatment with NX218 (2 mg/kg IP once a day for 28 days from day 11 to day 38 (group 3)) on Aβ₂₅₋₃₅-induced short-term memory deficits in mice. Data are expressed as a percentage of alternation over time. n is equal to 5-6 per group. ns (non-significant), **p < 0.01, ***p < 0.001 vs. the Aβ₂₅₋₃₅ vhc group (group 2), One-Way ANOVA followed by Dunnett’s test

With a 120-day follow up, NX218-treated mice at 2 mg/kg (Groups 3 and 4) showed a full and sustained restoration without any efficacy loss contrary to DPZ (Group 5) which is efficient only until D36.

Mice with already established pathology and cognitive deficits treated transiently from D11 to D38 with NX218 peptide (Group 3) completely recovered from memory alterations observed in YM. Moreover, the benefits have been maintained until D120 without re-administration of NX peptide, highlighting its disease-modifying effect (targeting of the pathogenic pathways of the disease to deeply modify or reverse the progression of the pathology (i.e. cognitive deficits)) rather than a symptomatic effect which is transient (as with marketed drugs such as DPZ).

Following DPZ resistance after 4 weeks of treatment (37% of alternation), NX218 treatment (Group 5) completely rescued memory loss for up to 17 weeks (between 53% and 80% of alternation depending on the time and dose of NX peptide administered).

NX218 + DPZ treated mice (Group 6) displayed a full and sustained restoration of spatial short-term working memory impairment (between 71% and 80% of alternation along the study versus 35-49% for vehicle-treated mice and 73-80% for Scramble-Aβ group) for up to 17 weeks without efficacy loss.

### Example 7: Pharmacokinetics in Animals

Preliminary in vitro experiments demonstrated that NX210 is rapidly converted into NX218 by oxidation in rat plasma. Therefore NX210 PK in animals is followed by the measurement of its cyclic form NX218. First a preliminary PK study in rat was performed in order to validate the method for detecting NX218 in plasma then translated to monkey by performing a more robust PK study with repeated experiments. All PK studies were performed with NaCl 0.9% as vehicle.

### Preliminary PK Study in Rat

In the 4 rats tested, NX218 rapidly decreased in concentration and became impossible to quantify from 3 hours after slow bolus IV injection of 49 mg/kg of NX210 (data not shown). This study demonstrated identification of NX218 in animal plasma was feasible.

### PK Study in Monkey

In the 3 monkeys tested, data were reproducible after repeated testing at different days (D22, D37 and D51), after bolus IV injection of 10 mg/kg of NX210. The concentration of NX218 rapidly decreased up to 30 minutes after the injection (FIG. 8). The half-life was assessed to approximately 12 minutes (Table 13), so in the same range as the one observed for rat and dog (data not shown), with a high consistency for repeated administrations.

TABLE 13

PK data after NX210 IV injection in monkey

| Study Day | Cmax | Tmax | AUCo-t | AUC_{0-inf} | CL | t_{½} | V_{SS} |
|---|---|---|---|---|---|---|---|
| | (ng/mL) | (min) | (min*ng/mL) | (min*ng/mL) | (mL/min/kg) | (min) | (mL/kg) |
| 22 (4300), 37 (4301 and 4302) or 51 (4300, 4301 and 4302) | 19300 | 0 | 57600 | 63900 | 190 | 12.7 | 93.6 |

AUC: Area Under Curve, CL: total clearance, Cmax: maximum concentration, t1/2: terminal elimination half-life, Tmax: time to reach Cmax, Vss: Volume of distribution at steady state
Summary statistics of mean monkey plasma PK parameters for NX218 (± standard deviation when available)

### Example 8: Pharmacokinetics in Human Healthy Subjects

As in animals; PK study of NX210 in Human Healthy subjects was conducted following the measurement of its cyclic form NX218. Six healthy subjects were administered a 10 mg/kg dose through an IV infusion lasting 12 minutes. t_{½} was assessed based on the data from 3 patients (see table below). The half-life was assessed to approximately 19 minutes (Table 14), so in the same range as the one observed for animals.

TABLE 14

Plasma PK parameters of NX210 after single IV administration of 10 mg/kg dose

| | Cmax (ng/m L) | Tmax (min) | AUClas t (min*ng/mL) | AUC∞ (min*ng/mL ) | t_{½} (min) | Kel (I/min) |
|---|---|---|---|---|---|---|
| N | 6 | 6 | 6 | 3a | 3a | 3a |
| AM | 582 | 9.00 | 4970 | 5930 | 20.0 | 0.0348 |
| SD | 297 | 2.45 | 2530 | 2740 | 2.15 | 0.0035 2 |
| Min | 185 | 6.00 | 1470 | 2800 | 18.6 | 0.0308 |
| Median | 575 | 10.00 | 5400 | 7060 | 19.0 | 0.0365 |
| Max | 1040 | 12.00 | 7850 | 7920 | 22.5 | 0.0372 |
| CV% | 51 | 27 | 51 | 46 | 11 | 10 |
| GM | 510 | 8.69 | 4290 | 5390 | 20.0 | 0.0347 |

AM=Arithmetic Mean; SD=Standard Deviation; Min=Minimum; Max=Maximum; GM=Geometric Mean;
CV : variation coefficient
Kel : clearance rate constant
AUC: Area Under Curve
Cmax: maximum concentration
t1/2: terminal elimination half-life
Tmax: time to reach Cmax
CV%=Arithmetic CV which is equal to SD/AM*100;
a:There are three subjects with AUClast/ AUC∞ ratio<0.80 or the adjusted R2 value < 0.80 therefore, their Kel, t_{½} and AUC∞ are not reportable and are not included in the summary statistics.

### REFERENCES

Maurice T, Lockhart BP, Privat A. Amnesia induced in mice by centrally administered β-amyloid peptides involves cholinergic dysfunction. Brain Res. 1996 Jan 15;706(2):181-93.

Maurice T, Su TP, Privat A. Sigma1 (σ1) receptor agonists and neurosteroids attenuate β25-35-amyloid peptide-induced amnesia in mice through a common mechanism. Neuroscience. 1998 Mar;83(2):413-28.

Maurice T, Mustafa MH, Desrumaux C, Keller E, Naert G, de la C García-Barceló M, Rodriguez Cruz Y, Garcia Rodriguez JC. Intranasal formulation of erythropoietin (EPO) showed potent protective activity against amyloid toxicity in the Aβ25-35 non-transgenic mouse model of Alzheimer’s disease. J Psychopharmacol. 2013 Nov;27(11): 1044-57.

Jackson S, Ham RJ, Wilkinson D. The safety and tolerability of donepezil in patients with Alzheimer’s Disease. Br J Clin Pharmacol. 2004 Nov;58 Suppl 1: 1-8.

Meunier J, leni J, Maurice T. The anti-amnesic and neuroprotective effects of donepezil against amyloid β25-35 peptide-induced toxicity in mice involve an interaction with the σ1 receptor. BrJ Pharmacol. 2006 Dec;149(8):998-1012.

Homma A, Imai Y, Tago H, Asada T, Shigeta M, Iwamoto T, Takita M, Arimoto I, Koma H, Takase T, Ohbayashi T. Long-Term Safety and Efficacy of Donepezil in Patients with Severe Alzheimer’s Disease. Dement Geriatr Cogn Disord. 2009;27(3):232-9.

1. A method of treating a tauopathv in a subject in need thereof, the method comprising administering to the subject, through a systemic route, a therapeutic amount of apeptide of amino acid sequence
X1-W-S-A1-W-S-A2-C-S-A3-A4-C-G-X2 (SEQ ID NO: 1)
in which :
A1, A2, A3 and A4 consists of amino acid sequences consisting of 1 to 5 amino acids,
X1 and X2 consists of amino acid sequences consisting of 1 to 6 amino acids; or X1 and X2 are absent;
it being possible for the N-terminal amino acid to be acetylated, for the C-terminal amino acid to be amidated, or the N-terminal amino acid to be acetylated and the C-terminal amino acid to be amidated.

2. The method of claim 1, wherein the peptide is of amino acid sequence
W-S-A1-W-S-A2-C-S-A3-A4-C-G (SEQ ID NO: 2)
in which:
A1, A2, A3 and A4 consists of amino acid sequences consisting of 1 to 5 amino acids.

3. The method of claim 1, wherein the peptide is a linear peptide or an oxidized peptide with the cysteines appearing on the peptide formula of SEQ ID NO: 1 forming a disulfide bridge, or a mixture of both linear and oxidized peptide.

4-16. (canceled)

17. The method of claim 2, wherein the peptide is a linear peptide or an oxidized peptide with the cysteines appearing on the peptide formula of SEQ ID NO: 2 forming a disulfide bridge, or a mixture of both linear and oxidized peptide.

18. The method of claim 1, wherein
A1 is chosen from G, V, S, P and A,
A2 is chosen from G, V, S, P and A,
A3 is chosen from R, A and V, and/or
A4 is chosen from S, T, P and A.

19. The method of claim 18, wherein
A1 is chosen from G, S,
A2 is chosen from G, S,
A3 is chosen from R, V, and/or
A4 is chosen from S, T.

20. The method of claim 1, wherein A1 and A2 are independently chosen from G and S, and/or A3-A4 is chosen from R-S or V-S or V-T or R-T.

21. The method of claim 1, wherein the peptide is of a sequence selected from the group consisting of sequences SEQ ID NO: 3- 63.

22. The method of claim 1, wherein the peptide is of sequence SEQ ID NO: 3, under linearized form, under cyclized form or a mixture of both.

23. The method of claim 1, wherein the tauopathy is selected from the group consisting of Alzheimer’s Disease (AD); Progressive Supranuclear Palsy (PSP); Tau positive Fronto-Temporal Dementia; dementia with Lewy bodies; corticobasal degeneration; Niemann-Pick type C disease; chronic traumatic encephalopathy; and postencephalitic parkinsonism.

24. The method of claim 1, wherein the tauopathy is Alzheimer’s disease (AD).

25. The method of claim 1, wherein the peptide induces reducing or disrupting tau aggregation in a subject, reducing tau protein in a subject, and/or reducing the level of phosphorylated tau protein.

26. The method of claim 1, wherein the peptide is administered to the patient via a route selected from the group consisting of the intravenous, intraperitoneal, intranasal, subcutaneous, intramuscular, sublingual, and oral routes.

27. The method of claim 1, wherein the subject is also treated with a sufficient amount of an acetylcholinesterase inhibitor.

28. The method of claim 27, wherein the acetylcholinesterase inhibitor is DPZ.

29. A pharmaceutical composition comprising at least one SCO-Spondin derived peptide and an acetylcholinesterase inhibitor, and a pharmaceutically acceptable vehicle, carrier or excipient.

30. The composition of claim 29, wherein the acetylcholinesterase inhibitor is DPZ.

31. A method of treating a tauopathy in a subject in need thereof, the method comprising administering to the subject, through a systemic route, a therapeutic amount of a composition comprising a peptide of sequence SEQ ID NO: 3, under linearized form, under cyclized form or a mixture of both, and a pharmaceutically acceptable vehicle or excipient.

32. A method of treating a tauopathy in a subject in need thereof, the method comprising administering to the subject, through a systemic route, a therapeutic amount of a peptide of sequence SEQ ID NO: 3, under linearized form, under cyclized form or a mixture of both, reducing or disrupting tau aggregation in said subject, reducing tau protein in said subject, and/or reducing the level of phosphorylated tau protein in said subject.
